(19) United States
(12) Patent Application Publication
(10) Pub. No.: US 2005/0069932 A1
(43) Pub. Date: Mar. 31, 2005

(54) Analyte evaluating device, and method for evaluating analyte

(75) Inventors: Kenji Arinaga, Kawasaki (JP); Ulrich Rant, Garching (DE); Shozo Fujita, Kawasaki (JP); Tsuyoshi Fujihara, Kawasaki (JP); Marc Tornow, Garching (DE); Gerhard Abstreiter, Garching (DE)

Correspondence Address: ARMSTRONG, KRATZ, QUINTOS, HANSON & BROOKS, LLP, 1725 K STREET, NW, SUITE 1000, WASHINGTON, DC, 20006, US

(73) Assignee: FUJITSU LIMITED, Kawasaki (JP)

(21) Appl. No.: 10/940,746

(22) Filed: Sep. 15, 2004

(30) Foreign Application Priority Data
Sep. 25, 2003 (JP) 2003-334271
Mar. 3, 2004 (JP) 2004-058376
Aug. 18, 2004 (JP) 2004-238696

(51) Int. Cl. C12Q 1/68; C12M 1/34; G01J 3/30

(52) U.S. Cl. 435/6; 435/287.2; 356/317

(57) ABSTRACT

In an analyte evaluating device comprising a carrier body that can be bound with an analyte having a fluorescence-labeled part that can emit fluorescence by light received when the distance between the fluorescence-labeled part and the carrier body is enlarged, at least one factor selected from the group consisting of a light irradiation angle, a light irradiation intensity, a light irradiation area, a fluorescence detection angle, a fluorescence detection area, the shape of the carrier body, the surface area of the carrier body, a salt concentration in a medium for use in the detection, and the adhesion density of analytes on the carrier body, is made to be adjustable. A high sensitivity is realized. Evaluation is possible without introducing fluorescence-labeled parts. Evaluation for a tiny amount of sample is possible. It is also possible to evaluate objects in a mixed state. Miniaturized, complex, and integrated devices are possible.

### CROSS-REFERENCE TO RELATED APPLICATIONS

This application is based upon and claims the benefit of priority from the prior Japanese Patent Application No. 2003-334271, filed on Sep. 25, 2003, the prior Japanese Patent Application No. 2004-058376, filed on Mar. 3, 2004 and the prior Japanese Patent Application No. 2004-238696, filed on August 18, 2004, the entire contents of which are incorporated herein by reference.

### BACKGROUND OF THE INVENTION

1. Field of the Invention

The present invention relates to the technology for evaluating an evaluation object represented by a biochip and a DNA chip.

2. Description of the Related Art

The human genome project that have advanced since the beginning of 1990's is a multinational effort in which each country takes a responsibility for part of the work to decode the whole human genetic codes, and it was announced in the summer of 2000 that the draft version of decoding was completed. It is expected that what kind of function each sequencing position for the decoded human genome sequencing information is related with, will be clarified as the functional genomic science and structural genomic science develop in future.

This human genome project has brought a great change in paradigm for scientific technologies and industries in relation with life science. For example, diabetes mellitus has been classified according to the condition of the disease that the blood glucose level is elevated, and regarding the causes of the manifestation, classification has been made into type I (being unable to produce insulin in the body), type II (being unable to control the amount of insulin in the body), etc., based on how much the insulin productivity is in the body of a patient.

A human genome project presents us all of the information of amino acid sequencing structures of proteins such as enzymes and receptors in relation with detection, synthesis, decomposition, and other regulations of blood glucose and insulin, and the information of the DNA sequencing of genomes in relation with control of the amount of such proteins present.

Using such information should make it possible to classify diabetes mellitus as a phenomenon that the blood glucose level is not regulated in a normal manner, into subtypes, based on which of the respective proteins in relation with a group of processes such as detection, synthesis and decomposition of blood glucose and insulin, are in disorder, and accordingly, appropriate diagnosis and treatment should become possible.

In particular, genome-based drug discovery for developing a medicine for a specific protein based on the human genome sequencing has been promoted energetically. It is now expected that time will come when genome-based drugs are administered based on the understanding of the state of such a group of the proteins functionally related with each other in order to alleviate symptoms and to cure a disease.

However, the technology for simply and conveniently measuring the amounts of such a group of proteins that are functionally related with each other, is still in the developing stage as a proteome analysis technology. One measurement method using two-dimensional electrophoresis in combination with mass spectrometry has been established. However, this method requires relatively large-scale apparatuses, and therefore, development of new technologies is needed to clinically ascertain the conditions of a disease of a patient, for example, in a laboratory or at the bedside of a patient in a hospital.

With such a need at the background, studies called micro-Total Analysis System (μ-TAS) and Lab-on-a-chip have attracted interest. These technologies provide microscopic devices obtained by forming grooves of a micrometer size (microchannels) on a several-centimeter-square substrate of glass or silicone in order to perform chemical analyses or chemical reactions. Owing to the fact that liquid or gaseous samples are made to flow into microscopic flow channels (several hundred to several μm in width), advantages are given such as reduced amounts of the samples and wastes, high-speed processing, etc. Furthermore, there is a possibility to miniaturize even chemical plants. Thus, application of such technologies to biotechnology is being expected. It is to be noted that I-TAS is translated into Japanese as “Shusekika Kagaku Bunseki System” (Accumulated Chemical Analysis System), “Maikuro Kagaku-Seikagaku Bunseki System” (Microchemical-Biochemical Analysis System), etc. It is a chemical analysis system with miniaturized sensors, analyzers, or the like, integrating, on a chip, functions of devices for use in analytical chemical laboratories.

Among these, the biochip technologies represented by DNA chips (or DNA microarrays) attract attention as effective means for gene analysis. Biochips comprises substrates made of glass, silicon, plastics, etc. on the surface of which numerous different test substances of biomacromolecules such as DNAs and proteins, are highly densely arrayed as spots. They can simplify examination of nucleic acids and proteins in the fields of clinical diagnosis and pharmacotherapy (for example, see Japanese Unexamined Patent Application Publication No. 2001-235468 (paragraph numbers 0002-0009), and “Journal of American Chemical Society”, vol. 119, p. 8916-8920, 1997.

As test substances, DNAs and nucleotides are used, for example. Accordingly, in many cases biochips are called DNA chips.

When fragments of unknown DNAs or analytes are made to flow into such a DNA chip, targeted DNAs are captured by hybridization with the test substances, utilizing the property of DNAs that they are bound or combined with complementary DNAs. If a fluorescence-labeled part is attached to the unknown DNAs beforehand, the captured analytes are detected by the fluorescence signals from respective spots on the DNA chip. Thus, the state of from several thousand to tens of thousands of DNAs or RNAs in the analytes can be observed at once by analyzing the data on a computer.

In such a so-called DNA chip, fluorescent pigments are introduced during the amplification (multiplication) of DNAs or the targeted objects for measurement that has been performed previously by PCR (polymerase chain reaction), so that the amounts of DNAs in a specimen bound with complementary DNA strands or chains located in an array are measured quantitatively by the intensities of fluorescence.

However, amplification of proteins corresponding to the PCR is not possible. Furthermore, there is a problem that uniform introduction of fluorescence-labeled parts is not possible owing to the difference in reactivity between each protein and the pigment, if numerous types of proteins are present in a specimen as a mixture.

### SUMMARY OF THE INVENTION

Accordingly, it is an object of the present invention to solve the above-described problems and provide a new technology for evaluating evaluation objects such as proteins with a high sensitivity. Other objects and advantages of the present invention will become evident from the following explanations.

According to one aspect of the present invention, an analyte evaluating device is provided which comprises a carrier body that can be bound with an analyte having a fluorescence-labeled part that can emit fluorescence by light received when the distance between the fluorescence-labeled part and the carrier body is enlarged, the distance between the fluorescence-labeled part and the carrier body being variable by an external action; a light irradiation device for the fluorescence-labeled part to emit light; and a fluorescence detecting device for detecting the fluorescence emitted by the fluorescence-labeled part, wherein at least one factor selected from the group consisting of a light irradiation angle, a light irradiation intensity, a light irradiation area, a fluorescence detection angle, a fluorescence detection area, the shape of the carrier body, the surface area of the carrier body, a salt concentration in a medium for use in the detection, and the adhesion density of analytes on the carrier body, is adjustable.

By the present invention, an analyte evaluating device for evaluating evaluation objects such as proteins with a high sensitivity is realized. It is possible to perform the evaluation without introducing fluorescence-labeled parts or radioactive materials into the evaluation objects. Evaluation for a tiny amount of sample is also possible. It is also possible to perform the evaluation, even if there are various kinds of evaluation objects in a mixed state in a sample. Furthermore, miniaturized, complex, and integrated analyte evaluating devices are possible.

Preferable are that the distance between the fluorescence-labeled part and the carrier body can be varied by a responding part that is located on at least one of the analyte and the carrier body; that the external action is an electromagnetic or chemical action; particularly that the carrier body is an electrode and the electromagnetic action is realized by applying an electric potential difference between the electrode and a counter electrode; that the carrier body can be chemically bound with the analyte; that the carrier body has a Au layer on the surface; that the carrier body has an analyte binding part bound with the Au layer via a thiol group; that the analyte has an evaluation object binding part that has a property to specifically bind to at least one evaluation object selected from the group consisting of proteins, DNAs, RNAs, antibodies, natural or artificial single-stranded nucleotides, natural or artificial double-stranded nucleotides, aptamers, products obtained by limited decomposition of antibodies with a protease, organic compounds having affinity to proteins, biomacromolecules having affinity to proteins, complex materials thereof, positively or negatively charged ionic polymers, and arbitrary combinations thereof; particularly that the evaluation object is a protein; that the responding part can be charged positively or negatively; that the responding part comprises at least one material selected from the group consisting of proteins, DNAs, RNAs, antibodies, natural or artificial single-stranded nucleotides, natural or artificial double-stranded nucleotides, aptamers, products obtained by limited decomposition of antibodies with a protease, organic compounds having affinity to proteins, biomacromolecules having affinity to proteins, complex materials thereof, positively or negatively charged ionic polymers, and arbitrary combinations thereof; particularly that the responding part comprises a natural or artificial single-stranded nucleotide, or a natural or artificial double-stranded nucleotide; that the responding part comprises a Fab fragment or (Fab)₂ fragment of an antibody; that the responding part comprises a fragment derived from an IgG antibody, or a fragment derived from a Fab fragment or (Fab)₂ fragment of an IgG antibody; that the responding part comprises an aptamer; that the light irradiation area is not less than the surface area of the carrier body, and the fluorescence detection area can be in the range of 80 to 120% of the surface area of the carrier body; that the light irradiation area is not less than the surface area of the carrier body, and the fluorescence detection area can be not more than half of the surface area of the carrier body; that the carrier body is bound with the analyte; that a plurality of the same type or different types of carrier bodies are installed; and that a plurality of the same type or different types of analytes are installed.

Also preferable are that the carrier body is an electrode; that the responding part is a wiry formation having a diameter of not more than 100 nm that can be positively or negatively charged, is expandable and shrinkable, and is fixed on the electrode; that at least one factor selected from the group consisting of an applied direct-current (DC) voltage, an applied alternate-current (AC) voltage, a frequency of the applied voltage, an electric potential at the center of the AC voltage, the length of the wiry formation, and the rate of adhesion of wiry formations on the carrier body is adjustable; and that the wiry formation is fixed onto the electrode by the physical or chemical adsorption.

According to another aspect of the present invention, a method for evaluating an analyte using an analyte evaluating device according to the above-described aspects is provided wherein: the analyte is made to be bound with the carrier body; the distance between the fluorescence-labeled part and the carrier body is varied by an external action; a light is emitted from the light irradiation device; the fluorescence emitted by the fluorescence-labeled part is detected from the fluorescence detecting device; and at least one factor selected from the group consisting of a light irradiation angle, a light irradiation intensity, a light irradiation area, a fluorescence detection angle, a fluorescence detection area, the shape of the carrier body, the surface area of the carrier body, a salt concentration in a medium for use in the detection, and the adhesion density of the analytes on a carrier body, is adjusted.

By the present invention, a method for evaluating an analyte with a high sensitivity is realized for evaluation objects such as proteins. It is possible to perform the evaluation without introducing fluorescence-labeled parts or radioactive materials into the evaluation objects. Evaluation for a tiny amount of sample is also possible. It is also possible to perform the evaluation, even if there are various kinds of evaluation objects in a mixed state in a sample.

Preferable are that at least one of the ratio of the light irradiation area to the surface area of the carrier body, and the ratio of the fluorescence detection area to the surface area of the carrier body, is adjusted; that the light irradiation area is made to be not less than the surface area of the carrier body, and the fluorescence detection area is made to be in the range of 80 to 120% of the surface area of the carrier body; that the light irradiation area is made to be not less than the surface area of the carrier body, and the fluorescence detection area is made to be not more than half of the surface area of the carrier body; that the salt concentration in the medium is adjusted to not more than 1 M; that the salt concentration in the medium is adjusted to not more than 100 mM; that the adhesion density of the analyte on the carrier body is adjusted in consideration of the size and length of the analyte, so that the analyte or a combination of the analyte and an evaluation object should not encounter a steric hindrance on the carrier body; that the analyte is made to be bound with the evaluation object before the analyte is made to be bound with the carrier body; that an electrode is used as the carrier body, and an electromagnetic action is realized by applying, between the electrode and a counter electrode, an electric potential difference having a value selected from the group consisting of a constant value, a pulse value, a stepwisely changing value and a periodically changing value, or a combination thereof; and that at least one physical property selected from the group consisting of presence or absence of fluorescence emission, the rate of increase in the fluorescence intensity, the rate of decrease in the fluorescence intensity, the peak fluorescence intensity, the rate of change of the peak fluorescence intensity, frequency properties of the rate of change in the fluorescence intensity and a cut-off frequency of the rate of change in the fluorescence intensity is measured; that the evaluation is performed, using the relationship between at least one physical property selected from the group consisting of the rate of increase in the fluorescence intensity, the rate of decrease in the fluorescence intensity, the peak fluorescence intensity, the rate of change of the peak fluorescence intensity, frequency properties of the rate of change in the fluorescence intensity and a cut-off frequency of the rate of change in the fluorescence intensity, and at least one property selected from the group consisting of a light irradiation angle, a light irradiation intensity, a light irradiation area, a fluorescence detection angle, a fluorescence detection area, the shape of the carrier body, the surface area of the carrier body, a salt concentration in a medium for use in the detection, the adhesion density of the analytes on the carrier body and an applied electric potential difference.

Also preferable are that the carrier body is an electrode, and the responding part is a wiry formation having a diameter of not more than 100 nm that can be positively or negatively charged, is expandable and shrinkable, and is fixed on the electrode; that the analyte is evaluated by making the wiry formation come near or move away from the carrier body by applying a sine-wave or rectangular-wave AC electric field; that at least one factor selected from the group consisting of an electric charge of the wiry formation or a substance adhered to the wiry formation, an electrostatic capacity thereof, an applied DC voltage, an applied AC voltage, a frequency of the applied voltage, an electric potential at the center of the AC voltage, the length of the wiry formation, and the rate of adhesion of wiry formations on the carrier body is adjusted; that the electric potential at the center of the AC voltage is adjusted for the carrier body to have a zero voltage; that the electric potential at the center of the AC voltage is changed in a stepwise manner or continuously; and that the electric potential at the center of the AC voltage is changed in the range between a low electric potential region where a stronger fluorescence signal starts to be obtained, and a high electric potential region where the fluorescence signal is rapidly weakened, both of which appear when the electric potential at the center of the AC voltage is changed in a stepwise manner or continuously.

By the present invention, an analyte evaluating device and an analyte evaluating method with a high sensitivity are realized for evaluation objects such as proteins.

It is also possible to perform the evaluation without introducing fluorescence-labeled parts or radioactive materials into the evaluation objects. It is also possible to perform the evaluation, even if the amount of the analyte is small. It is also possible to perform the evaluation, even if there are various kinds of evaluation objects in a mixed state in a sample. Furthermore, miniaturized, complex, and integrated analyte evaluating devices and excellent analyte evaluating methods utilizing the devices can be provided.

It is to be noted that the “analyte” in the present invention refers to an object to be detected and evaluated for finally evaluating an evaluation object with an analyte evaluating device. A case in which an evaluation object itself is an analyte as well as a case in which an analyte bound with an evaluation object is detected and evaluated is included in the category of the present invention. An analyte evaluating device has a function to comprehend an evaluation object by detection and evaluation of an analyte, and is a concept corresponding to a biochip or a DNA chip.

However, a case in which an analyte is either included or not included as explained later is also included in the category of the analyte evaluating device according to the present invention. Those devices in which a plurality of analyte evaluating devices are arranged, for example, in a dense array, are also included in the category of the analyte evaluating device according to the present invention. Furthermore, those devices integrated with other devices having other functions are also included in the category of the analyte evaluating device according to the present invention.

### BRIEF DESCRIPTION OF THE DRAWINGS

FIG. 1 is a model view illustrating how an analyte bound to a carrier body expands or shrinks, emitting or extinguishing fluorescence;

FIG. 2-A is a model view illustrating an analyte bound with a carrier body;

FIG. 2-B is a model view illustrating an analyte detached from a carrier body;

FIG. 3-A is a model view illustrating an analyte bound with a carrier body and shrunk;

FIG. 3-B is a model view illustrating an analyte bound with a carrier body and laid down;

FIG. 3-C is a model view illustrating an analyte expanded from a carrier body;

FIG. 4 is a graph showing change of fluorescence intensity with the passage of time;

FIG. 5 is another graph showing change of fluorescence intensity with the passage of time;

FIG. 6 is an combination of graphs for explaining the effect of a salt, regarding an analyte evaluating device according to the present invention;

FIG. 7 is a graph for explaining the relationship between the fluorescence intensity peak value and the salt concentration;

FIG. 8 is a model view illustrating how an analyte bound to a carrier body expands or shrinks, emitting or extinguishing fluorescence;

FIG. 9 is a model view illustrating how light is irradiated onto analytes that are on the way of detaching from a carrier body to cause fluorescence, and the fluorescence is observed, in an analyte evaluating device according to the present invention;

FIG. 10 is another model view illustrating how light is irradiated onto analytes that are on the way of detaching from a carrier body to cause fluorescence, and the fluorescence is observed, in an analyte evaluating device according to the present invention;

FIG. 11 is a graph showing change of fluorescence intensity with the passage of time;

FIG. 12 is another model view illustrating how light is irradiated onto analytes that are on the way of detaching from a carrier body to cause fluorescence, and the fluorescence is observed, in an analyte evaluating device according to the present invention;

FIG. 13 is another graph showing change of fluorescence intensity with the passage of time;

FIG. 14 is a model view illustrating an example of arrangement of an analyte evaluating device according to the present invention;

FIG. 15 is a model view illustrating another example of arrangement of an analyte evaluating device according to the present invention;

FIG. 16 is a graph showing the behavior of a normalized fluorescence intensity when the salt concentration is changed finely;

FIG. 17 is a graph showing change of a normalized fluorescence intensity with the passage of time after application of a potential difference at a certain salt concentration;

FIG. 18 is a graph showing potential differences applied in a stepwise manner with the passage of time, regarding an analyte evaluating device according to the present invention;

FIG. 19 is a graph explaining the influence of salt concentration, regarding an analyte evaluating device according to the present invention;

FIG. 20 is a combination of graphs showing an example of a series of changes in fluorescence intensity;

FIG. 21 is a combination of graphs showing changes of fluorescence intensity, etc. when a stepwisely increasing DC bias added with a weak AC was applied as an AC electric field;

FIG. 22 is a graph showing the effect of the salt concentration in a medium on the rate of change of fluorescence intensity;

FIG. 23 is a graph showing the effect of the surface adhesion rate of nanowires onto an electrode on the rate of change of fluorescence intensity;

FIG. 24 is a combination of graphs showing the result of observation of the change of fluorescence intensity when a rectangular-wave AC voltage of ±200 mV (0 mV-400 mV) based on 200 mV was applied to an electrode;

FIG. 25 is a combination of graphs showing the result of measurements when an AC voltage was applied for a long time;

FIG. 26 is a graph showing the effect of salt concentration in a medium on the rate of change of fluorescence intensity;

FIG. 27 is a graph showing the effect of the surface adhesion rate of nanowires onto an electrode on the rate of change of fluorescence intensity;

FIG. 28 is a combination of graphs showing changes of fluorescence intensity, etc. when a stepwisely increasing DC bias added with a weak AC was applied as an AC-electric field;

FIG. 29 is a graph showing how the change of fluorescence intensity is dependent on the frequency when a sine-wave AC bias was used as an AC electric field;

FIG. 30 is a graph showing the change of fluorescence intensity when a small amount of streptavidin that can be specifically bound to biotins introduced as complementary strands was added to an aqueous solution;

FIG. 31 is a graph showing how the rate of change in the fluorescence intensity is dependent on the frequency before and after the hybridization, when a sine-wave AC bias was used as an AC electric field;

FIG. 32 is a graph showing how the dependence of the rate of change in the normalized fluorescence intensity on the frequency (cut-ff frequency) changes before and after the hybridization, when a sine-wave AC bias was used as an AC electric field; and

FIG. 33 is a graph showing how the dependence of the rate of change in the normalized fluorescence intensity on the frequency (cut-ff frequency) changes in the presence and absence of a biotin-streptavidin mixture.

### DESCRIPTION OF THE PREFERRED EMBODIMENTS

For the purpose of solving the above-described problems, in Japanese patent applications No. 2002-297934, 2002-297941, etc., disclosed are technologies in which proteins are specifically determined quantitatively without applying labeling reactions such as fluorescence labeling as well as element technologies applicable to array chip technologies through which information useful from the viewpoint of proteome for comprehending proteins as a group is obtained.

It is an object of the present invention to improve the sensitivity in the evaluation in such technologies and to make it easier for such technologies to be applied as element technologies. It is to be noted that the evaluation according to the present invention means detection of the presence and absence of an evaluation object as well as quantitative measurement.

An analyte evaluating device according to the present invention comprises a carrier body that can be bound with an analyte having a fluorescence-labeled part that can emit fluorescence by light received when the distance between the fluorescence-labeled part and the carrier body is enlarged, the distance between the fluorescence-labeled part and the carrier body being variable by an external action, a light irradiation device for the fluorescence-labeled part to emit light, and a fluorescence detecting device for detecting the fluorescence emitted by the fluorescence-labeled part.

In this device, an evaluation object can be evaluated by binding the analyte to the carrier body, realizing a state of the fluorescence-labeled part being extinguished by the quenching effect, and then, making the fluorescence-labeled part emit light by enlarging the distance between the fluorescence-labeled part and the carrier body by an external action in order to observe the behavior of increasing and decreasing of the emitted light.

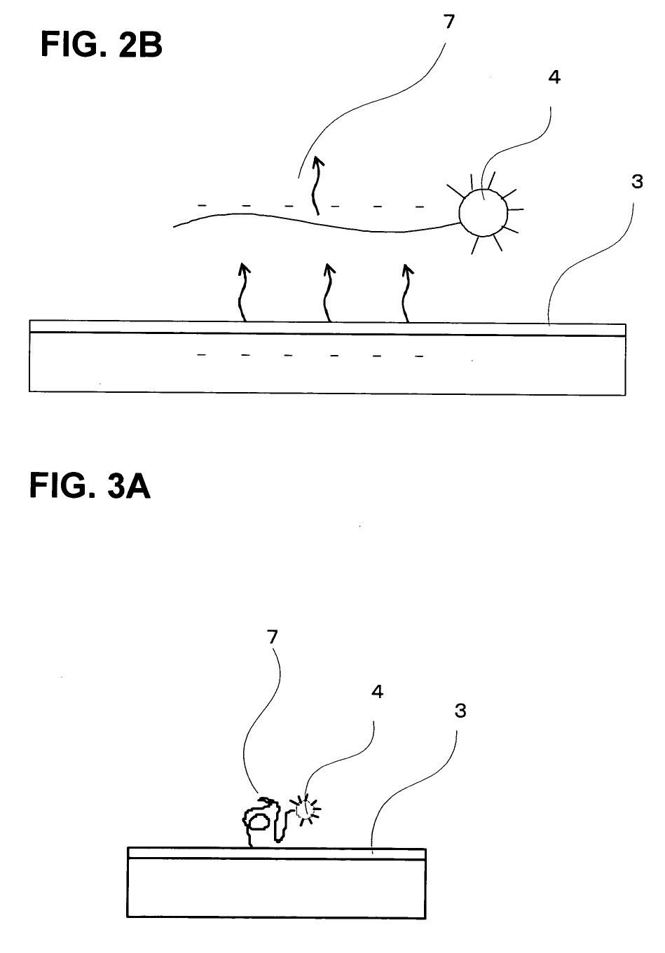

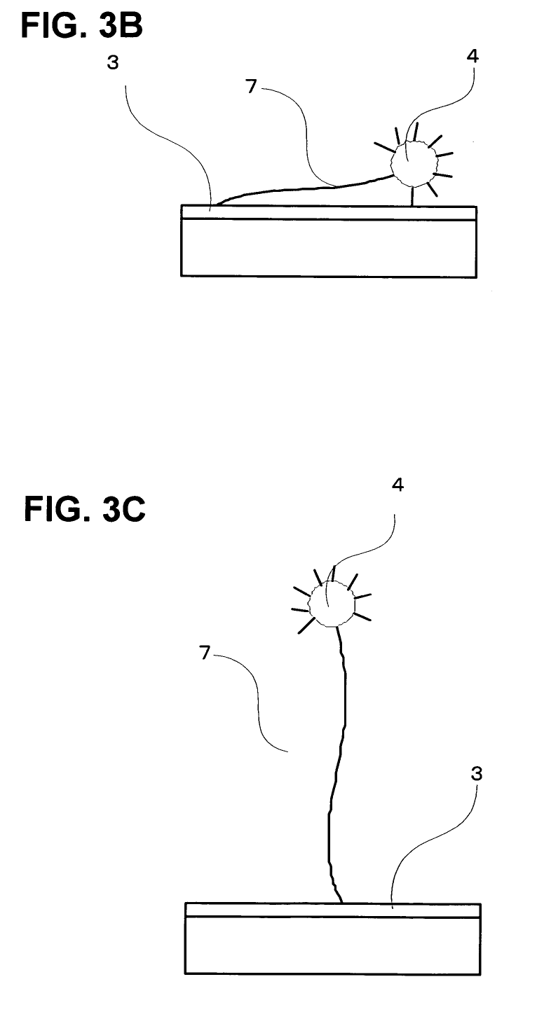

It is preferable that the distance between the fluorescence-labeled part and the carrier body can be varied by a responding part that has a function of detaching an analyte from the carrier body and/or expanding/shrinking an analyte, in response to an external action. Whether the responding part is located on the analyte or on the carrier body, the purpose can be achieved. It is to be noted that enlarging the distance between the fluorescence-labeled part and the carrier body can be realized by the detachment of the analyte from the carrier body or by expanding the responding part. FIG. 2A is a model view illustrating a state of an analyte 7 being bound with a carrier body 3 by adsorption, and FIG. 2B is a model view illustrating a state of an analyte 7 being detached from a carrier body 3. Furthermore, FIG. 3A is a model view illustrating a state of an analyte 7 being bound with a carrier body 3 and shrunk, FIG. 3B is a model view illustrating a state of an analyte 7 being bound with a carrier body 3 and laid down, and FIG. 3C is a model view illustrating a state of an analyte 7 being expanded from a carrier body 3. Regarding the case in which an analyte is detached from a carrier body, it is possible to consider that the responding part is located either on the analyte or on the carrier body or on both of them.

An evaluation object may be an analyte itself. It may also be a material that can be bound with an analyte or a material bound with an analyte as will be explained later. An evaluation object is preferably selected from the group consisting of proteins, DNAs, RNAs, antibodies, natural or artificial single-stranded nucleotides, natural or artificial double-stranded nucleotides, aptamers, products obtained by limited decomposition of antibodies with a protease, organic compounds having affinity to proteins, biomacromolecules having affinity to proteins, complex materials thereof, and arbitrary combinations thereof. Examples of the complex materials in the present invention include combined materials from DNAs and negatively-charged polymers or the like, and combined materials from the above-described materials and other materials. An evaluation object is preferably a protein.

Hereupon, the “nucleotide” according to the present invention is any one selected from the group consisting of mononucleotides, oligonucleotides and polynucleotides, or a mixture thereof. Such materials are often negatively charged. Single-stranded nucleotides and double-stranded nucleotides can be used. They can also be specifically bound with analytes through hybridization. Proteins, DNAs and nucleotides can be used as a mixture. The biomacromolecules include those derived from living organisms, those processed from materials derived from living organisms, and synthesized molecules, too.

Hereupon, the above-described “products” are those obtained by limited decomposition of antibodies with a protease, and can comprise anything, as long as they conform to the gist of the present invention, including Fab fragments or (Fab)₂ fragments of antibodies, fragments derived from Fab fragments or (Fab)₂ fragments of antibodies, derivatives thereof, etc.

As an antibody, monoclonal immunoglobulin IgG antibodies can be used, for example. Fab fragments or (Fab)₂ fragments of IgG antibodies can be used as fragments derived from IgG antibodies, for example. Furthermore, fragments derived from those Fab fragments or (Fab)₂ fragments can also be used. Examples of applicable organic compounds having affinity to proteins are enzyme substrate analogs such as nicotinamide adenine dinucleotide (NAD), enzyme activity inhibitors, neurotransmission inhibitors (antagonist), etc. Examples of biomacromolecules having affinity to proteins are proteins that can act as a substrate or a catalyst for proteins, element proteins constituting molecular composites, etc.

Any action that can vary the distance between the carrier body and the fluorescence-labeled part may be used as an external action. Electromagnetic or chemical actions are practical, and accordingly preferable.

For example, an electromagnetic action can be realized by using an electrode as a carrier body, installing a counter electrode, and giving a potential difference between these electrodes. The electromagnetic action can be realized by providing a potential difference having either one of a constant value, a pulse value, a stepwisely changing value, a periodically changing value, or a combination thereof, between the carrier body and the counter electrode.

By employing such various types of potential difference, it is possible to evaluate the behaviors of expansion/shrinking, detachment from the carrier body, diffusion, etc. of analytes under various conditions. It is also possible to separate those that are relatively hard to detach from the carrier body from those that are relatively easily detached in order to perform evaluation.

Any chemical actions can be used, including scission of chemical bonds such as covalent bonds and coordinate bonds that are existing, as well as preventing or furnishing ionic, hydrophobic, or polar interactions.

In response to the above-described various evaluation conditions, it is useful, for evaluating analytes, to measure at least one physical property selected from the group consisting of presence or absence of fluorescence emission, the rate of increase in the fluorescence intensity, the rate of decrease in the fluorescence intensity, the peak fluorescence intensity, the rate of change of the peak fluorescence intensity, frequency properties of the rate of change in the fluorescence intensity and a cut-off frequency of the rate of change in the fluorescence intensity.

By these evaluations, the presence or absence of binding of analytes and/or kinds of bound analytes and/or the amounts of bound analytes can be detected thorough the relationship between properties such as the rate of increase in the fluorescence intensity, the rate of decrease in the fluorescence intensity, the peak fluorescence intensity, the rate of change of the peak fluorescence intensity, frequency properties of the rate of change in the fluorescence intensity and a cut-off frequency of the rate of change of the peak fluorescence intensity, and properties such as a light irradiation angle, a light irradiation intensity, a light irradiation area, a fluorescence detection angle, a fluorescence detection area, the shape of a carrier body, the surface area of a carrier body, a salt concentration in a medium for use in the detection, the adhesion density of analytes on a carrier body and an applied electric potential difference. Furthermore, the presence or absence of binding of biomacromolecules with the evaluation object binding part and/or kinds of bound biomacromolecules and/or the amounts of bound biomacromolecules can be detected.

A fluorescence-labeled part that emits or extinguishes fluorescence may be attached by a covalent bond to an evaluation object as its part. It may also be attached by a covalent bond to an analyte as its part before binding with an evaluation object, or may be included in a nucleotide or the like as shown in an example in which it is inserted (by intercalation) between adjacent complementary bonds, or integrated by substitution as a part of a nucleotide or the like. A fluorescence-labeled part is preferably located near the tip of an analyte.

A fluorescence-labeled part is selected from materials that are excited by the action of light and emit fluorescence. Examples suitable for use as a fluorescence-labeled part according to the present invention are indocarbocyanine 3 (trademark Cy3), etc.

Any material can be used as an analyte as long as it can be bound with a carrier body, and does not contradict the gist of the present invention. Preferable are those having, before binding with evaluation objects, a fluorescence-labeled part that can emit fluorescence by receiving light when the distance between the fluorescence-labeled part and the carrier body is enlarged.

It is preferable that the analyte has an evaluation object binding part having a property of specifically binding with an evaluation object. Evaluation is made possible by making evaluation objects such as proteins bind with analytes via this evaluation object binding part, without applying fluorescence-labeling reactions or the like.

Such an evaluation object binding part preferably has a property of specifically bound to the above-described evaluation objects. There is no particular limitation to the type and the site of binding. However, it would be better to avoid binding with a particularly weak binding force.

A responding part has a function of being able to vary the distance between the fluorescence-labeled part and the carrier body by an external action. Varying the distance between the fluorescence-labeled part and the carrier body can be caused, as described before, by expansion/shrinking of the responding part as well as by detaching the analyte from the carrier body. For the purpose of varying the distance between the fluorescence-labeled part and the carrier body by an electromagnetic action, it is preferable that the responding part is positively or negatively charged.

Such a responding part preferably comprises at least one material selected from the group consisting of proteins, DNAs, RNAs, antibodies, natural or artificial single-stranded nucleotides, natural or artificial double-stranded nucleotides, aptamers, products obtained by limited decomposition of antibodies with a protease, organic compounds having affinity to proteins, biomacromolecules having affinity to proteins, complex materials thereof, positively or negatively charged ionic polymers, and arbitrary combinations thereof, because, in many occasions, it is easy to perform expansion and shrinking as well as detachment from the carrier body, and to be specifically bound with an evaluation object by acting also as an evaluation object binding part. Examples of a responding part charged positively or negatively include positively charged DNAs (guanidine DNAs) by utilizing guanidide bonding in the main chain, and negatively charged natural nucleotides.

Hereupon, the above-described “product” is obtained by limited decomposition of antibodies with a protease, and as long as the gist of the present invention is met, anything including Fab fragments or (Fab)₂ fragments of antibodies, fragments derived from those Fab fragments or (Fab)₂ fragments of antibodies, derivatives thereof, etc. can be included.

As an antibody, a monoclonal immunoglobulin IgG antibody can be used for example. Fab fragments or (Fab)₂ fragments of IgG antibodies can also be used as fragments derived from IgG antibodies, for example. Furthermore, fragments derived from those Fab fragments or (Fab)₂ fragments can also be used. Examples of applicable organic compounds having affinity to proteins are enzyme substrate analogs such as nicotinamide adenine dinucleotide (NAD), enzyme activity inhibitors, neurotransmission inhibitors (antagonist), etc. Examples of biomacromolecules having affinity to proteins are proteins that can act as a substrate or a catalyst for proteins, element proteins constituting molecular composites, etc.

As a responding part, natural nucleotides and artificial nucleotides can be used. Artificial nucleotides include completely artificial nucleotides and those derived from natural nucleotides. In some cases, use of artificial nucleotides may be advantageous in raising the sensitivity and improving the consistency of detection.

A responding part may also be a single-stranded nucleotide or a double-stranded nucleotide that is a pair of complementarily-related single-stranded nucleotides. In many cases, single-stranded nucleotides are preferable owing to the ease of expansion and shrinking, and double-stranded nucleotides are preferable so as to make them rise and lie down on the carrier body. It is possible to use a different nucleotide for each electrode. Nucleotides with one or more residual groups are acceptable. That is, mononucleotides are acceptable.

Monoclonal antibodies and products obtained by limited decomposition with a protease can also be used for a responding part. They are useful, since bonds created by the reactions similar to antigen-antibody reactions can be utilized, and they can act also as evaluation object binding parts.

For the responding part, it is also preferable to use monoclonal antibodies, Fab fragments or (Fab)₂ fragments of monoclonal antibodies, or fragments derived from Fab fragments or (Fab)₂ fragments of monoclonal antibodies. It is to be noted that the fragments derived from Fab fragments or (Fab)₂ fragments of monoclonal antibodies mean fragments obtained by fragmenting Fab fragments or (Fab)₂ fragments of monoclonal antibodies, or derivatives thereof.

Furthermore, it is more preferable to use, as a responding part, IgG antibodies, Fab fragments or (Fab)₂ fragments of IgG antibodies, or fragments derived from IgG antibodies, or Fab fragments or (Fab)₂ fragments of IgG antibodies. It is to be noted that the fragments derived from Fab fragments or (Fab)₂ fragments of IgG antibodies mean fragments obtained by fragmenting Fab fragments or (Fab)₂ fragments of IgG antibodies, or derivatives thereof. Aptamers are also preferable. The reason is that those with a smaller molecular weight provide better detection sensitivity in general.

It is to be noted that not only cases in which the fluorescence-labeled part, evaluation object binding part, responding part and analyte are clearly distinct from each other but also cases in which part or the whole of one or more of them is also part or the whole of another or others, are included in the present invention. When the responding part comprises at least one material selected from the group consisting of proteins, DNAs, RNAs, antibodies, natural or artificial single-stranded nucleotides, natural or artificial double-stranded nucleotides, aptamers, products obtained by limited decomposition of antibodies with a protease, organic compounds having affinity to proteins, biomacromolecules having affinity to proteins, complex materials thereof, positively or negatively charged ionic polymers, and arbitrary combinations thereof, it may also have a part that functions as an evaluation object binding part in many cases.

Any material can be used as a carrier body according to the present invention, and there is no particular limitation to its shape, as long as it can be bound with an analyte, can vary the distance from a fluorescence-labeled part by an external action, and does not contradict the gist of the present invention. In this case, any type of binding can be utilized as long as it does not contradict the gist of the present invention, including biological binding, electrostatic binding, physical adsorption, chemical adsorption, etc., as well as chemical bonding such as covalent bonding and coordinate bonding.

For example, glasses, ceramics, plastics, metals, etc. can be carrier bodies according to the present invention. Those having a structure part (analyte binding part) that can be bound with an analyte on the surface, can also be used. The carrier body may be single-layered, or multi-layered. It may also has a structure other than layers.

Any material can be arbitrarily chosen for the carrier body depending on the purpose, but Au is particularly preferable. When a biomacromolecule is used as an analyte, it is easy to fix it onto a carrier body.

When an electromagnetic action is used as the external action, it is reasonable to use the whole or part of the carrier body as an electrode. An electroconductive material itself can be used as a carrier body. It is also possible to install a layer of an electroconductive material on the surface of a glass, ceramic, plastic, metal or the like. As such an electroconductive material, any material can be used including simple metal substances, alloys, laminates thereof, etc. Noble metals of which Au is representative, are preferably used owing to their chemical stability.

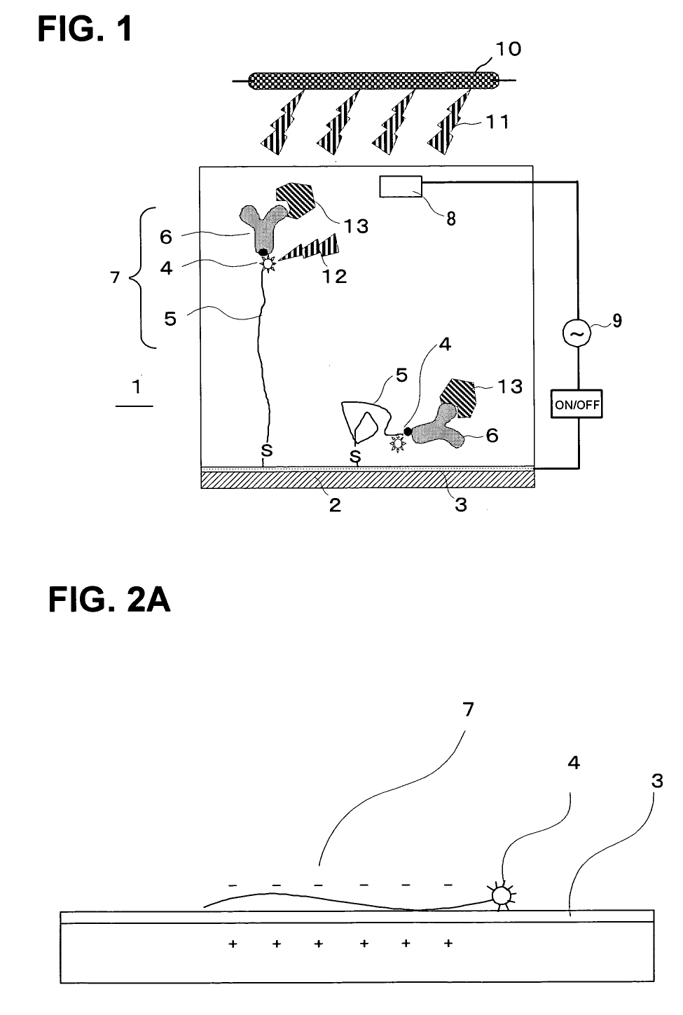

When the carrier can be bound with an analyte without specifically forming an analyte binding part, it is not necessary to install an analyte binding part on the surface. Taking a case in which an analyte comprises a nucleotide, and can be bound with a Au layer directly via its thiol group for example, there is an analyte evaluating device 1 as shown in FIG. 1 wherein analytes 7 are bound with a Au electrode (carrier body 3) installed on a sapphire substrate 2, the analytes having a fluorescence-labeled part 4, a responding part 5 having a natural single-stranded oligonucleotide structure, and an evaluation object binding part 6, and the binding being established by the reaction with the polished Au electrode at room temperature for 24 hours. “S” which is located in the lower portion of the single-stranded oligonucleotide structure represents that the analyte 7 is directly bound with the Au electrode 3 via a thiol group. It is also possible to use known metals other than Au for an electrode surface to be bound to a thiol group. In FIG. 1, a Fab fragment of a monoclonal immunoglobulin IgG is fixed at the tip of the oligonucleotide strands, as the evaluation object binding part 6 having a property to be specifically bound with an evaluation object.

On the left of FIG. 1, a state of an analyte being expanded, is illustrated. On the right, a state of an analyte being shrunk, is illustrated. The analyte 7 in the shrunk state can be expanded by applying a specific potential difference between the Au electrode 3 and a counter electrode 8 through an external electric field applying device 9. In this example, the distance between the fluorescence-labeled part and the carrier body varies not by detaching the analyte from the carrier body, but by the expansion and/or shrinking of the responding part that is part of the analyte. In this case, the analyte is left as being fixed on the carrier body, and accordingly, the measurement is advantageous since it can be performed continuously and can be repeated many times, compared with the evaluation utilizing the detaching phenomenon.

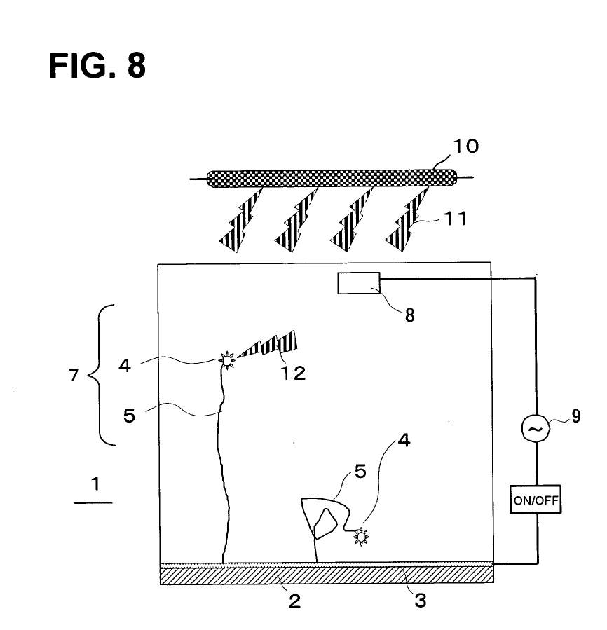

In this state, fluorescence 12 is provided as light 11 is irradiated from a light irradiation device 10. In FIG. 1, an evaluation object 13 is bound with the evaluation object binding part 6. When an analyte itself is an evaluation object, emission or extinction of fluorescence is evaluated without binding the analyte to an evaluation object, as shown in FIG. 8. This case does not require an evaluation object binding part.

In FIG. 1, the thio group and the fluorescence-labeled part were introduced onto a single-stranded oligonucleotide beforehand. It is preferable that the thiol group and the fluorescence-labeled part be introduced onto the end of a single strand. When the thiol group is introduced onto the 5′ end of the strand, it is preferable to introduce the fluorescence-labeled part onto the 3′ end of the strand, and when the thiol group is introduced onto the 3′ end of the strand, it is preferable to introduce the fluorescence-labeled part onto the 5′ end of the strand. In this example, the oligonucleotide strand was fixed onto the circular Au electrode having a diameter of 1 mm.

When an analyte binding part is installed as part of a carrier body, any material can be used as the analyte binding part, as long as it can be bound with an analyte. Examples are molecules that can be bound with an analyte via chemical bonding or intermolecular force. If the analyte binding part can expand/shrink or can detach an analyte, it can also act as a responding part. In this case, detaching from an analyte occurs not necessarily at a position where the analyte binding part is bound with the analyte. For example, in a preferred embodiment wherein an analyte binding part is bound to a Au layer via a thiol group, detaching at a thiol group may be possible. Accordingly, it goes without saying that a case in which the position of detaching of an analyte from a carrier body may be different from the position where the carrier body is bound with the analyte, also belongs to the scope of the present invention in general.

Hereupon, although it is generally ideal that the binding between a carrier body and an analyte is quantitative, there can be binding with a significantly large dissociation constant. If the dissociation constant is too large, the amount of bond may gradually decrease, for example, during washing with a buffer solution. From this viewpoint, it is generally preferable that the dissociation constant in the binding between a carrier body and an analyte be not mot than 10⁻⁵.

When such a carrier body is immersed in an aqueous solution, it is possible to expand the analyte binding part as a DC electric field is, for example, applied between the carrier body and a counter electrode in the aqueous solution, and the analyte binding part shrinks spontaneously as the electric field is cut off.

Or, even if a counter electrode is absent, it is possible to make a negatively charged analyte binding part expand by Coulomb repulsion, when a negative electric field is applied to the carrier body (electrode).

Therefore, when the analyte binding part is regarded as a responding part according to the present invention, a fluorescence-labeled part in the analyte bound to the analyte binding part can be made to emit and extinguish fluorescence. As a result, a fluorescence-labeled part near the electrode that had extinguished fluorescence starts to emit fluorescence owing to the fact that the fluorescence-labeled part moves away from the surface of the electrode sufficiently.

Any known materials may be used as the light irradiation device for the fluorescence-labeled part such as a fluorescent molecule to emit fluorescence, and the fluorescence detecting device for detecting fluorescence emitted from the fluorescence-labeled part. Use of one or more optical fibers is advantageous in many cases, since the devices should be applied to a microscopic area. Optical fibers with an inner diameter of about 1 μm to about 10 mm can be used.

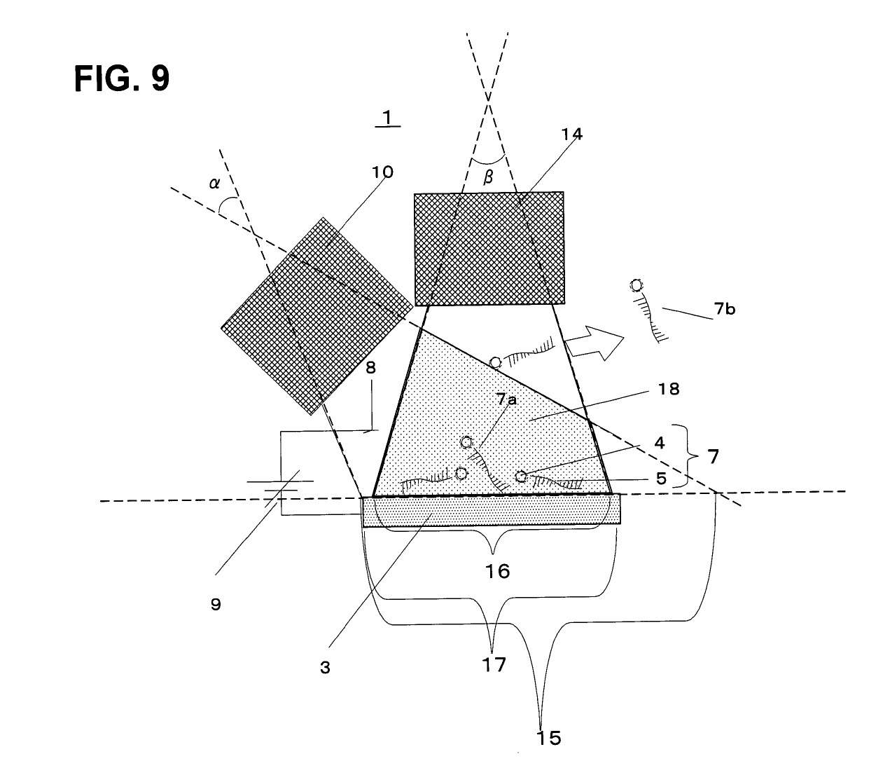

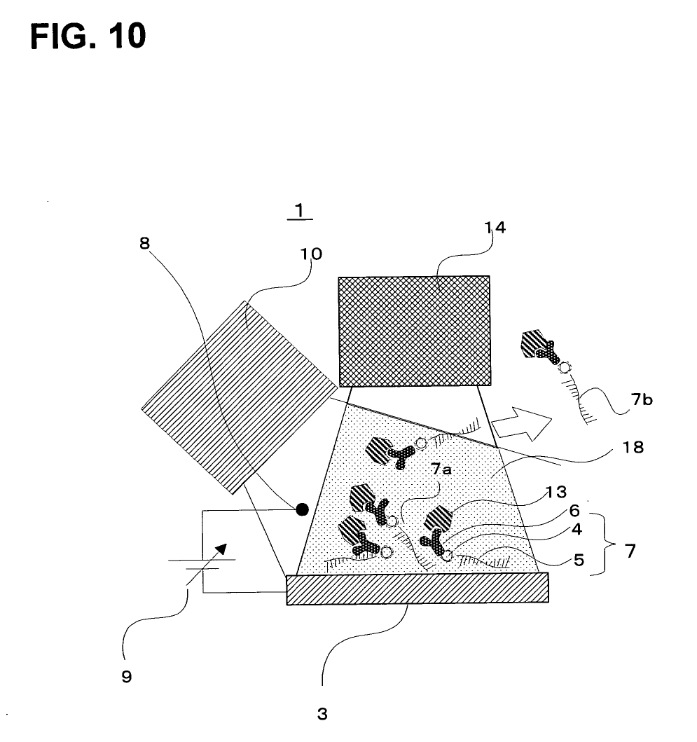

Next, a case is explained, using FIGS. 9 and 10 in which an electrode acts as a carrier body, an electromagnetic action is realized by providing a potential difference between the electrode and a counter electrode, and accordingly, analytes bound to a carrier body are detached from the carrier body, so that the fluorescence emitted as a result of the detachment is detected.

FIG. 9 is a model view illustrating that fluorescence is generated by irradiating light on analytes on the way of leaving a carrier body, and the fluorescence is detected. In FIG. 9, an analyte evaluating device 1 according to the present invention comprises an electrode 3 bound with analytes 7 having a fluorescence-labeled part 4 and a responding part 5, a counter electrode 8 in the aqueous solution (a medium), an external electric field applying device 9, a light irradiation device 10 comprising optical fibers, and a fluorescence detecting device 14 comprising optical fibers.

As shown in FIG. 9, by applying a potential difference between the electrode 3 and the counter electrode 8 with the external electric field applying device 9, the analytes 7 bound with electrode 3 are detached from the electrode 3. The increase and decrease of fluorescence during the course are detected with the fluorescence detecting device 14.

Hereupon, it is to be noted that FIG. 9 illustrates a case in which the analytes 7 themselves are the evaluation objects, and accordingly, evaluation object binding parts and evaluation objects bound with the evaluation object binding parts are not included in the figure. In contrast, FIG. 10 illustrates a case in which evaluation object binding parts 6 that are parts of the analytes 7 are bound with evaluation objects 13.

With the analyte evaluating device according to the present invention, at least one factor selected from the group consisting of a light irradiation angle, a light irradiation intensity, a light irradiation area, a fluorescence detection angle, a fluorescence detection area, the shape of the carrier body, the surface area of the carrier body, a salt concentration in a medium for use in the detection, and the adhesion density of analytes on the carrier body, is adjustable, and an evaluation object can be evaluated by adjusting these factors. In FIG. 9, the reference sign α refers to a light irradiation angle. the numeral 15 refers to a light irradiation area, the reference sign β refers to a fluorescence detection angle, the reference numeral 16 refers to a fluorescence detection area, and the reference numeral 17 refers to the surface area of the carrier body, each as a component in a cross-section.

When such a factor is adjustable, it is possible to change behaviors of the increase or decrease, the maximum, etc. of fluorescence. It is further possible to change such behaviors by changing the extent of the external actions and the way to provide such external actions. Accordingly, it is possible to perform evaluation of an evaluation object precisely and quickly through such changes of behaviors. It is to be noted that such adjustment be preferably performed before or during the use of the analyte evaluating device according to the present invention.

Adjustment of at least one of the ratio of the light irradiation area to the surface area of the carrier body, and the ratio of the fluorescence detection area to the surface area of the carrier body is a preferable example for the evaluation method of the analyte.

In illustrating the effect of the adjustment for example, FIG. 9 shows a case in which the light irradiation area is made to be not less than the surface area of the carrier body, and the fluorescence detection area is made to be on the same level as the surface area of the carrier body. The fluorescence detection area is preferably made to be in the range of 80 to 120% of the surface area of the carrier body. It is more preferably made to be in the range of 80 to 100% of the surface area of the carrier body.

In such cases, the analytes are expanded or detached from the carrier body by electrostatic repulsion when an electric field with an appropriate polarity is applied. This expansion or detachment makes the fluorescence-labeled part 4 which was extinguished by the quenching effect, emit fluorescence and the fluorescence is observed by the fluorescence detecting device 14.

As time passes further, the fluorescence decreases quickly and comes to disappear soon, since the analytes 7 shrink to the original state by the compensation effect of counter ions in the medium when they are expanded, and the analytes 7 move out of the light detection area 18 shown as a tetragon in FIG. 9, by diffusion in the aqueous solution, when they are detached. Analytes 7a exemplify analytes inside the light detection area 18, and analytes 7b exemplify analytes outside the light detection area 18. It was experimentally ascertained that, in such an occasion, the fluorescence intensity and the change of the fluorescence intensity are proportional to the amount and the rate of dispersion of analytes that are dispersing into an aqueous medium, as are related with the sizes of the carrier body and the fluorescence detecting device.

Adjustment of factors such as a light irradiation angle, a light irradiation intensity, a light irradiation area, a fluorescence detection angle, a fluorescence detection area, the shape of the carrier body, the surface area of the carrier body, and a salt concentration in a medium for use in the detection, is particularly useful when an analyte can be detached from a carrier body. It is because more information can be obtained, since it is easy for the fluorescence-labeled part to emit fluorescence through the detachment, and to extinguish fluorescence owing to dispersion of the analyte out of the light detection area.

In such a case, by setting a light irradiation angle of the light irradiation device 10 so as to allow the incident light to irradiate all over the entire surface of the electrode 3, it is possible to observe the fluorescence of the analytes of all the surface of the carrier body, so a high level of the fluorescence intensity is realized. Also, by making the fluorescence detection area of the fluorescence detection device 14 on the same level as the surface are of the electrode 3, it is possible to start the observation at a time when substantially no analyte is present except in the light detection area, and accordingly, it is possible to realize a state that substantially no analytes that are entering into the light detection area by dispersion have not entered yet. Thus, it is possible to easily observe a state of analytes being dispersed from the light detection area.

When the time constant for detachment of analytes that are leaving a carrier body is represented by τᵣ, the time constant for diffusion of analytes that are diffusing out of the light detection area, τ_{d}, the amount of analytes which have detached from the carrier body, N, and the amount of analytes which can be observed in the light receiving area (that is, fluorescence detection area), M, the following approximate relationships (1) and (2) can be established.

$$\begin{matrix}\frac{ⅆ N}{ⅆ t} = - \frac{N}{{τ}_{r}} & ( 1 ) \\ \frac{ⅆ M}{ⅆ t} = \frac{N}{{τ}_{r}} - \frac{M}{{τ}_{d}} & ( 2 )\end{matrix}$$

Solving these equations for M results in equation (3), wherein No is the amount of analytes that were first combined with the carrier.

$$M ( t ) = \frac{{τ}_{d}}{{τ}_{r} - {τ}_{d}} {N}_{0} \{ exp ( - \frac{t - {t}_{0}}{{τ}_{r}} ) - exp ( - \frac{t - {t}_{0}}{{τ}_{d}} ) \} + const . \qquad ( 3 )$$

It is to be noted that though equation (3) is a very simple approximation, it can be directly compared with the fluorescence intensity behavior, since it is considered that proportionality is established between M and the fluorescence intensity as long as influences of the light quenching and the polarity of solvents are not large.

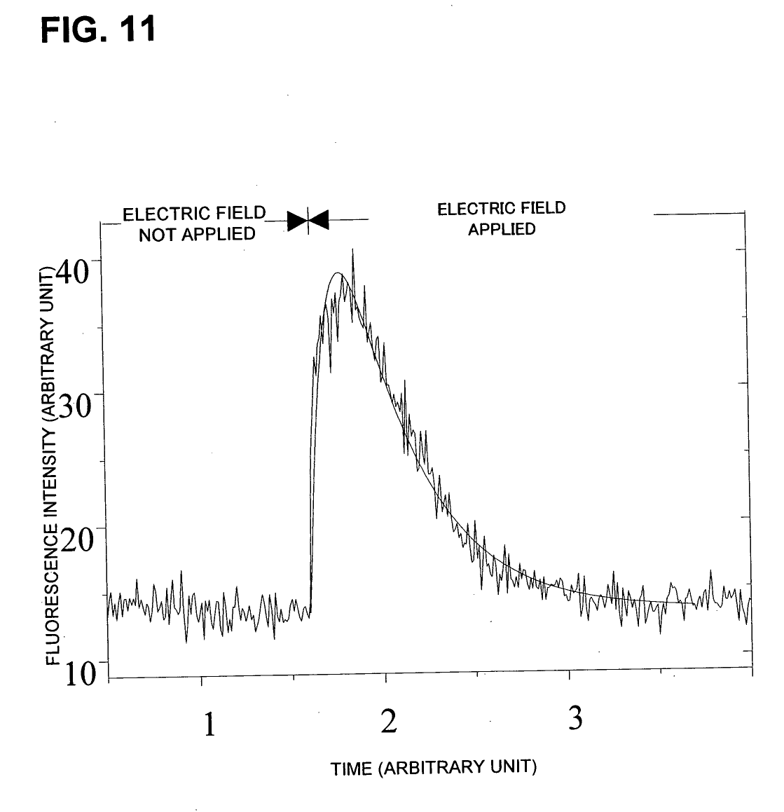

The result of comparison with the fluorescence intensity behaviors indicates a very good coincidence between the experimental values (broken line) and calculated values (thick solid line), as shown in FIG. 11. This result proves that an analyte evaluating device according to the present invention can evaluate the behavior of analytes as well as the behavior of evaluation objects combined with analytes, precisely, at a high sensitivity, and easily.

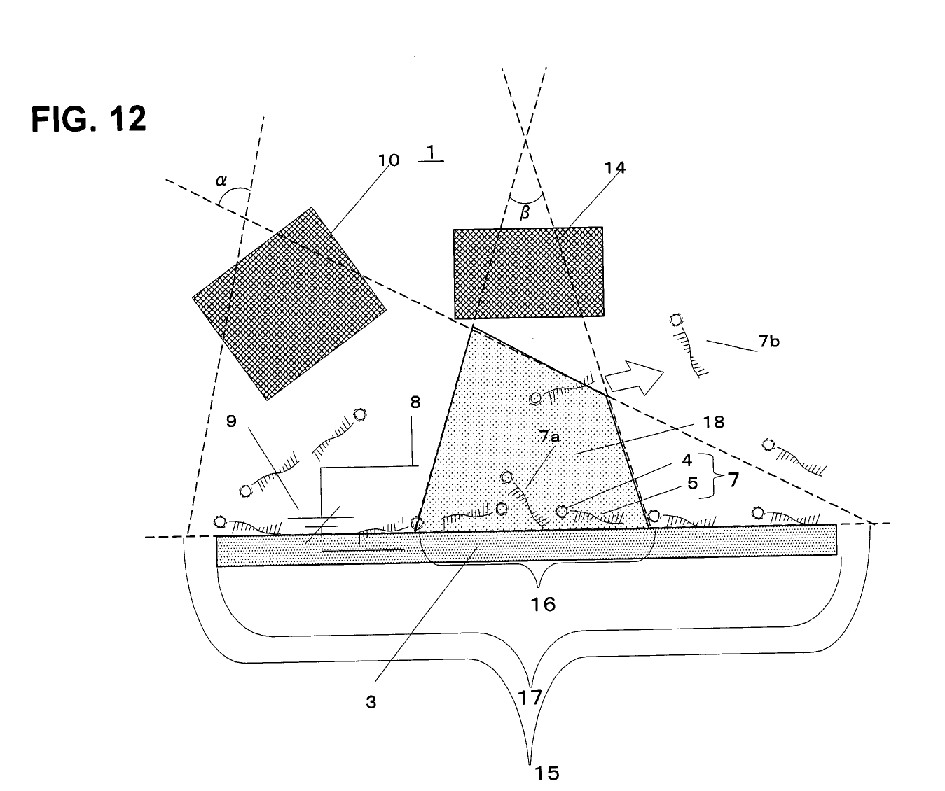

It is also useful, as shown in FIG. 12, that the light irradiation area 15 is not less than the surface area 17 of the carrier body, and the fluorescence detection area 16 is not more than half of the surface area 17 of the carrier body.

By employing these conditions, it is possible to set a light irradiation device 10 for the incident light to irradiate the entire surface of the electrode 3 so that fluorescence of analytes from the entire surface of the carrier body can be observed, resulting in a large fluorescence intensity. Furthermore, because the fluorescence detection area 16 is sufficiently smaller than the surface area 17 of the electrode 3, there are analytes also present outside of the light detection area. Accordingly, it is possible to realize a state that the number of analytes entering the light detection area 18 by diffusion is similar to the number of analytes exiting from the light detection area 18 by diffusion.

Therefore, a state that diffusion appears not to advance and a fluorescence intensity is saturated can be kept long, and the behavior of fluorescence reduction starts after a considerable duration of time.

In such a case, analytes that have been once electrostatically repulsed, spread outside of the light detection area by diffusion after a sufficiently long duration of time, for example, and accordingly, the detaching process by electrostatic repulsion and the diffusion process to the outside can be separately investigated. Furthermore, the value obtained by subtracting the background value from the fluorescence intensity at a saturated state (a normalized fluorescence intensity) is proportional to the amount of the detached analytes, and accordingly, the amount of the detached analytes can be compared by comparing these amounts.

In such a case, equations (4) and (5) can be each independently applied instead of equation (3).

$$\begin{matrix}\frac{ⅆ M}{ⅆ t} = \frac{M}{{τ}_{r}} & ( 4 ) \\ \frac{ⅆ M}{ⅆ t} = const . - \frac{M}{{τ}_{d}} & ( 5 )\end{matrix}$$

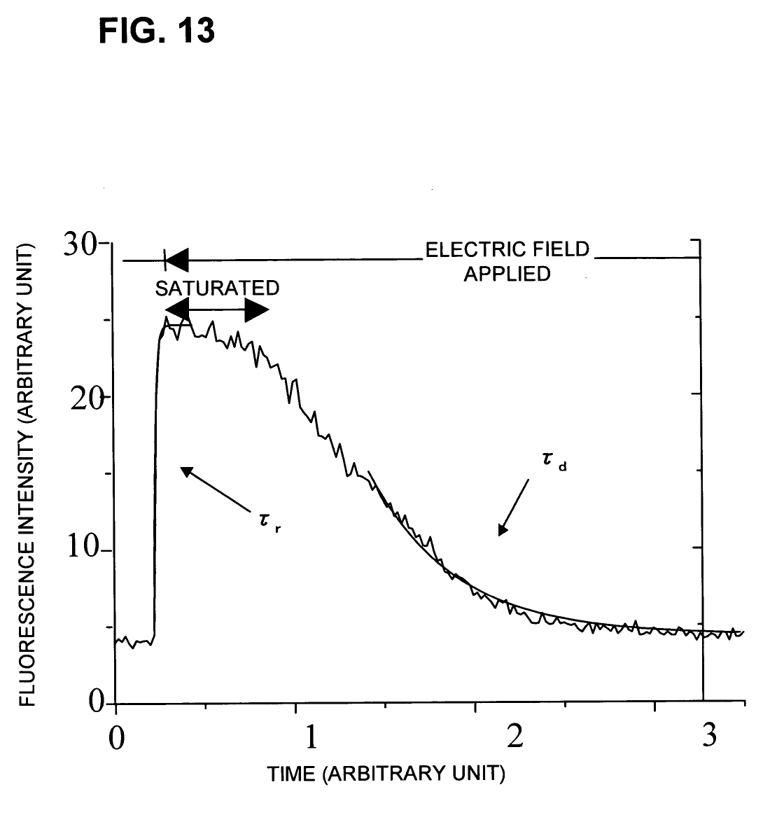

FIG. 13 shows the results of independent evaluations of the time constant for detachment, τᵣ, and the time constant for diffusion, τ_{d}. Experimental results represented by the broken line coincides very well with approximation represented by the thick solid line. This indicates that the time constant for detachment of analytes and the time constant for diffusion of analytes can be handled independently and the device can serve as a protein detection device greatly suitable for detecting and evaluating proteins.

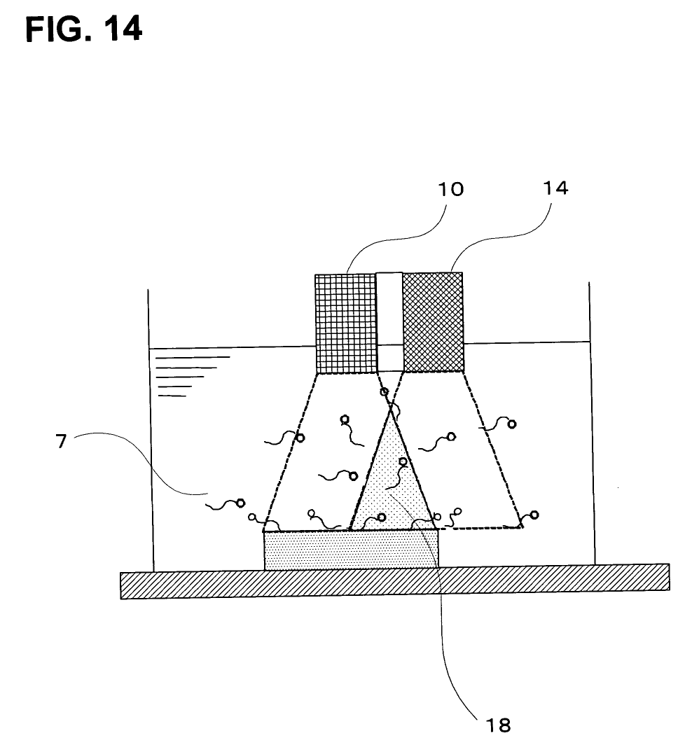

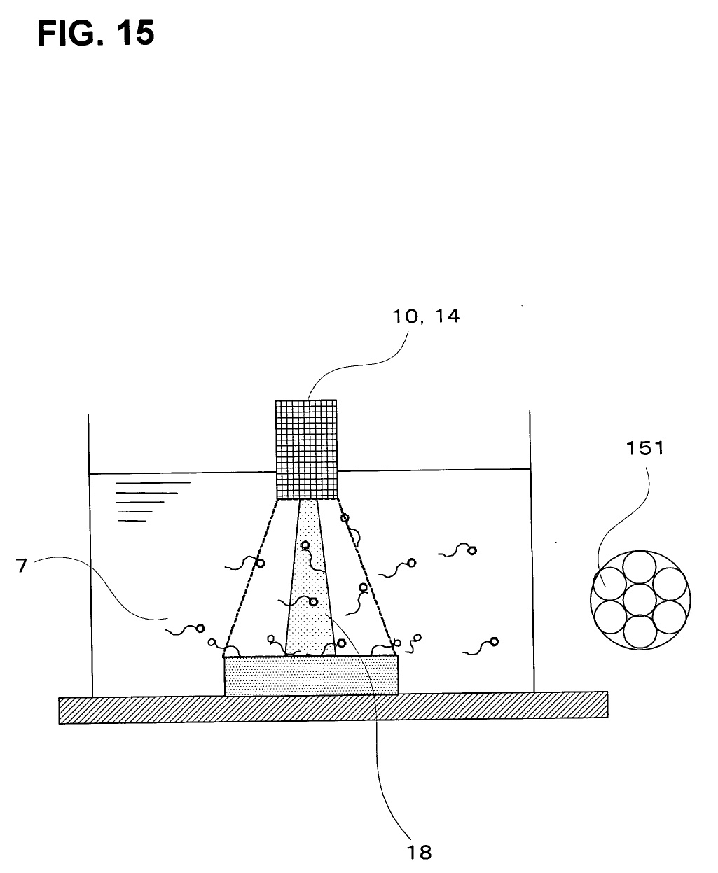

It is to be noted that in order to make the fluorescence detection area not more than half of the surface area of the carrier body, any arrangements can be employed including an arrangement as shown in FIG. 14, wherein a light irradiation device 10 and a fluorescence detection device 14 are aligned side by side, and an arrangement as shown in FIG. 15, wherein both the devices are integrated. Hereupon, FIG. 15 shows an example wherein a plurality of optical fibers 151 are bundled together for use.

The above are examples in which a light irradiation area and a fluorescence detection area are chosen appropriately. When evaluation of an evaluation object is performed, using an analyte evaluating device according to the present invention, there are other various complex conditions. In order to use an analyte evaluating device according to the present invention appropriately under such conditions, it is preferable to use controlling factors such as the light irradiation angle, the light irradiation intensity, the fluorescence detection angle, the shape of the carrier body and the surface area of the carrier body, as well as well as the light irradiation area and the fluorescence detection area.

In the detection of fluorescence according to the present invention, the salt concentration in the aqueous solution that is an environment (medium) for an analyte or an evaluation object to be bound with the analyte, is a useful controlling factor, too. A salt is added to prevent an evaluation object from aggregating. Inorganic salts, organic salts, or the like can be used as the salt, appropriately. As an inorganic salt, exemplified are sodium chloride, potassium chloride, magnesium chloride, etc.

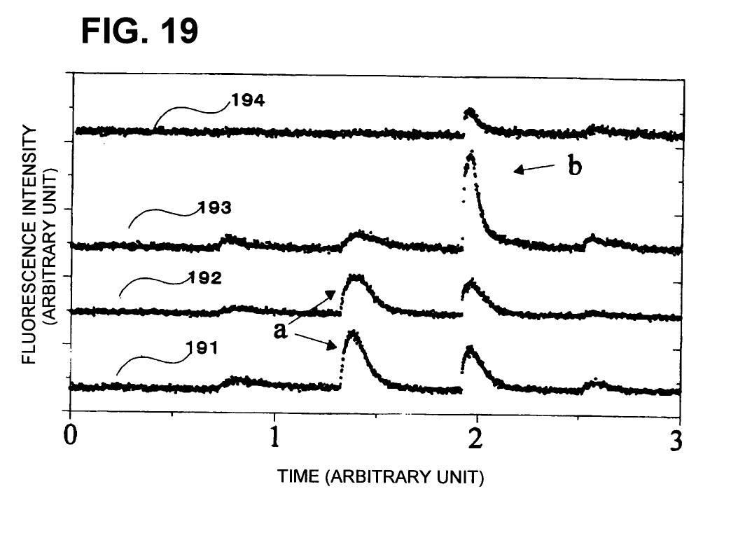

FIGS. 18 and 19 explain the effect of salt concentration on an analyte evaluating device 1 according to the present invention in which an analyte 7 comprising a responding part 5 having a fluorescence-labeled part 4 and a natural single-stranded oligonucleotide structure, and an evaluation object binding part 6 is bound to a carrier body 3 as shown in FIG. 1. FIG. 18 shows a change of the applied electric potential in a stepwise manner with the passage of time, and FIG. 19 shows the change of fluorescence intensity observed at the potential application with the passage of time. The data in FIG. 19 indicates the salt concentration in the increasing order of, numerals 191, 192, 193 and 194.

From FIGS. 18 and 19, it is shown that when the salt concentration is low, a relatively large change in fluorescence intensity is observed at a low electric potential difference as points a's in FIG. 19 indicates. Furthermore, when the salt concentration is raised, the fluorescence intensity can be observed at a high sensitivity at a specific salt concentration and a specific potential difference, as shown at point b in FIG. 19. Accordingly, it is possible to determine the kind of an analyte or an evaluation object bound with an analyte, by observing the fluorescence behavior while appropriately changing the salt concentration and the electric potential difference. Furthermore, detection at a relatively low potential difference and the increase of the detection sensitivity are also possible by setting the salt concentration appropriately.

It is to be noted that while the peak at point b in FIG. 19 increases up to a certain level as the salt concentration increases, it tends to decrease beyond the level. In order to avoid such a condition and to realize a condition in which the fluorescence is prevented from becoming small, the expansion or the detachment of an analyte from the carrier body is performed with a high efficiency, and the behavior is observed at a high sensitivity, it is sometimes preferable to control the salt concentration in an aqueous solution medium at not more than 1 M (mol/L). The concentration is more preferably not more than 100 mM (millimol/L), since the detection at a relatively low potential difference is possible at a high sensitivity, and electrolyzation of an aqueous solution medium and electrically-caused damage of analytes and evaluation objects can be restrained.

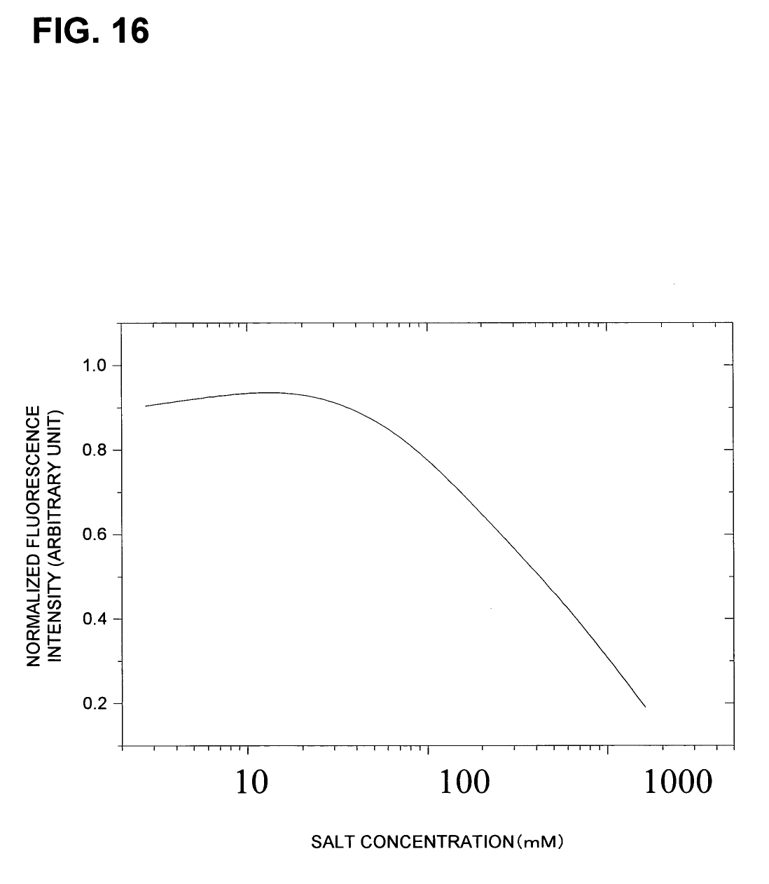

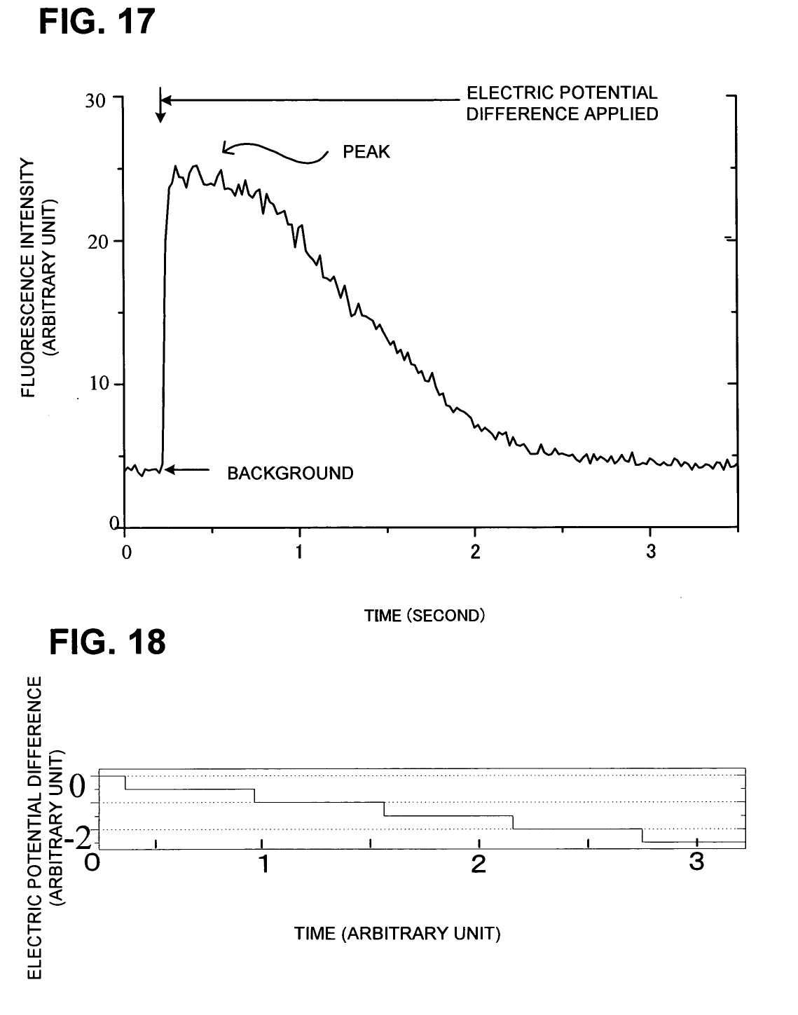

FIG. 16 shows a example of experimental results of normalized fluorescence intensity when the salt concentration is changed further finely, while fixing the applied electric potential difference. This figure is obtained by using the change of fluorescence intensity at a certain salt concentration with the passage of time after applying electric potential difference as shown in FIG. 17, subtracting the background from the peak of fluorescence intensity at a saturated state, and plotting the normalized fluorescence intensities proportional to amounts of the analytes that are detached from the electrode.

From FIG. 16, it is evident that the detachment of an analyte tends to be restrained at a specific salt concentration (in the neighborhood of 70-100 mM in the case of FIG. 16) or higher. Therefore, it is to be understood that it is useful to use an aqueous solution medium at a relatively low concentration of not more than 100 mM in order to maintain the sensitivity of a detection device using an analyte at a high level.

When an analyte is specifically bound with an evaluation object, detachment and diffusion of an analyte naturally change according to the mass and the electric charge of an evaluation object. Accordingly, the change in fluorescence intensity becomes different greatly. Therefore, by utilizing such changes, detection of evaluation objects such as proteins is possible at a high sensitivity with an analyte evaluating device according to the present invention.

Heretofore, the analyte evaluating device according to the present invention has been mainly explained on carrier bodies that can be bound with analytes and have not been bound yet. However, the analyte evaluating device according to the present invention is not limited to this, and those in which a carrier has already been bound with an analyte are also included in the scope of the present invention.

Also, neither the number nor the type of carrier is limited to one. An analyte evaluating device having a plurality of carriers of the same type or different types installed are included in the category of the present invention.

Similarly, an analyte evaluating device having a plurality of analytes of the same type or different types installed are included in the category of the present invention.

In an analyte evaluating device according to a certain embodiment of the present invention, an electrode acts as a carrier body, and a wiry formation having a diameter of not more than 100 nm acts as a responding part that can be positively or negatively charged, is expandable and shrinkable, and is fixed on the electrode. Accordingly, in the following embodiments, explanation will be made not on a case of an analyte that is leaving a carrier body, but on a case of a responding part that is expanding or shrinking. It goes without saying, hereupon, that the various embodiments that have been explained heretofore, can be applied to these embodiments, too, as long as they do not contradict with the gist of the present invention.

Any material can be arbitrarily chosen as a wiry formation (referred to as “nanowire”, hereafter) having a diameter of not more than 100 nm from the materials used for the above-described responding part as long as the diameter of the material is not more than 100 nm. However, any other wiry formation can be chosen as long as it has a diameter of not more than 100 nm. In such a case, the circle-equivalent diameter, i.e. a diameter of a circle having an equivalent area, may be used as the diameter if the material does not have a circular cross-section.

To be more concrete, enumerated are example from materials with atoms each being bound in series, to materials in a wiry shape that are formed by binding complicate molecules such as the above-described DNA's and polymers. Those that are formed by binding atoms in series may have a diameter of about 0.1 nm, and double-stranded DNA's may have a diameter of about 2 nm, for example. It is to be noted here that when modifying groups are attached, it is sufficient to consider only the diameter of the main chain.

The requirement of being wiry is satisfied, if the aspect ratio of the length to diameter is not less than 2:1, and there is no requirement for the shape. Various shapes including a tubular shape, a stripe shape, a coiled shape, a laminar shape, etc. can be utilized. In such a case, the aspect ratio can be determined as a value for the material being in a fully expanded state.

Those that are formed by binding plural —CH₂— units are examples of a nanowire that can be easily acquired. If 20 of the units are bound together, a nanowire is formed with a length of 2.68 nm when the —CH₂— units are in a expanded state, since the length of the C—C bond is 0.134 nm. In this case, the diameter of the nanowire is considered to be in the range of 0.3 to 0.5 nm, since the diameter is three to four times of the atomic diameter of carbon (C).

Ionic polymers bound with a group having an electric charge (such as COO⁻ and NH3⁺) may be used for nanowires to be positively or negatively charged, for example. However, any other suitable method can be employed. When molecules originally having an electric charge such as DNA's are bound into a wiry shape for use, such binding with a group having a charge is unnecessary. A single-stranded 12-mer DNA that will be described later makes a nanowire having a diameter of about 1.1 nm and a length of about 5 nm, when fully expanded.

It is possible to determine whether a nanowire is expandable or not by determining whether the evaluation of an analyte according to the present invention is possible, and it is not necessary to confirm that the material is actually expanded or shrunk. A behavior of change in the locational relationship with a carrier body that is caused by lying down, rising or the like of the nanowire, which are not generally regarded as included in the category of expanding/shrinking movements, and a behavior of change in the locational relationship with a carrier body that is caused by folding, bending or the like of the whole or part of the nanowire, can be included in the category of “expandable/shrinkable” of the present invention, as long as the evaluation of an analyte according to the present invention is possible. Carbon nanotubes are examples of these cases.

Any method can be employed for fixing a nanowire onto an electrode. Fixing by physical adsorption and fixing by chemical adsorption are examples.

In an analyte evaluating device wherein a nanowire is used, it is preferable that at least one factor selected from the group consisting of an applied DC voltage, an applied AC voltage, a frequency of the applied voltage, an electric potential at the center of the AC voltage, the length of the wiry formation, and the rate of adhesion of wiry formations on the carrier body is adjustable, in order to make possible the evaluation explained below.

When the above-described analyte evaluating device is used, it is preferable to evaluate an analyte by applying a sine-wave or rectangular-wave AC electric field so as to make the wiry formation come near or go away from the carrier body.

In this case, when at least one factor selected from the group consisting of the electric charge of a wiry formation or a substance attached to a wiry formation, its electrostatic capacity, an applied DC voltage, an applied AC voltage, a frequency of the applied voltage, an electric potential at the center of the AC voltage, the length of the wiry formation, and the rate of adhesion of wiry formations on the carrier body is adjusted, it is possible to obtain various information based on the molecular weight and character of analytes. A substance adhered to a wiry formation in this case may be the above-described evaluation object itself. Any substance that can change the information obtained from analytes may also be used.

For example, a nanowire expands or shrinks, when the charge of a substance attached to a nanowire receives electrostatic repulsion or electrostatic attraction created by an AC electric field applied to a carrier body with an external electric field applying device. Performing the evaluation by changing the electric charge of a wiry formation or a substance attached to a wiry formation, its electrostatic capacity, an applied DC voltage, an applied AC voltage, a frequency of the applied voltage, an electric potential at the center of the AC voltage, or the like, utilizing this phenomenon, can provide various information, and accordingly, more precise detection of the presence or absence of an evaluation object and quantitative determination of an evaluation object can be performed.

A device shown in FIG. 1 can be employed, for example, for an analyte evaluating device according to this embodiment using a fluorescence-labeled part for evaluation. In this case, the responding part 5 is a wiry formation (nanowire) having a diameter of not more than 100 nm that can be positively or negatively charged, is expendable and shrinkable, and is fixed to the electrode.

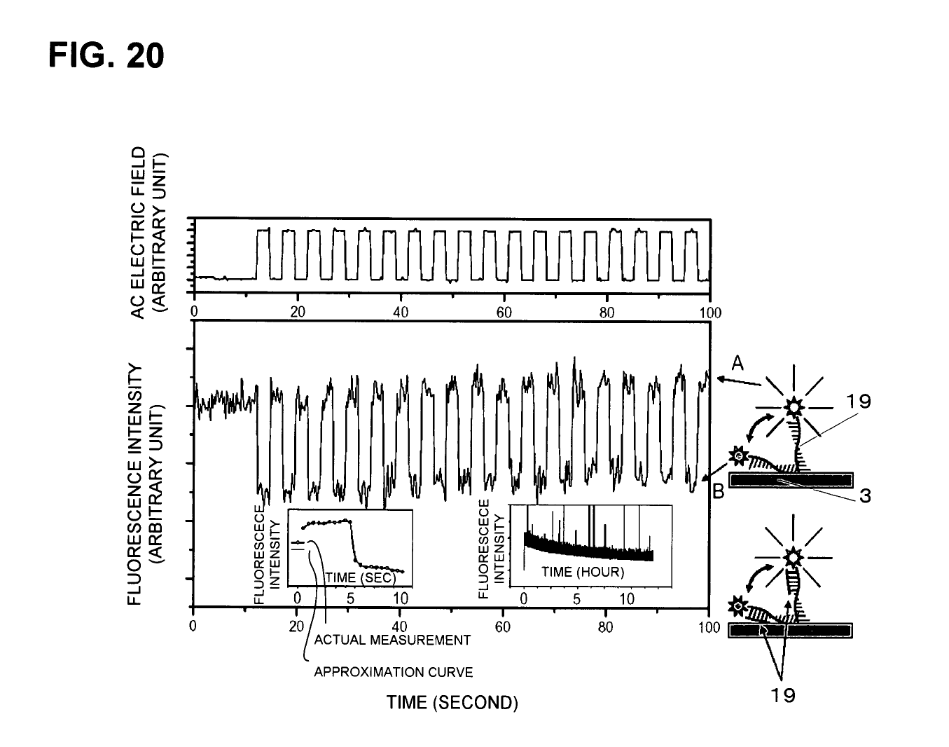

FIG. 20 shows an example of a series of changes in the fluorescence intensity in this case. The change with the passage of time in the fluorescence intensity observed when a rectangular-shape AC electric field as shown in the upper part of FIG. 20 is applied is shown in the lower part. A schematic view shown in the right-hand side illustrates how a nanowire expands and shrinks.

The state A with a large fluorescence intensity represents a state of a nanowire 19 being detached from an electrode 3 by electrostatic repulsion and the state B with a small fluorescence intensity represents a state of a nanowire 19 approaching the electrode 3 by electrostatic attraction. Evaluation of the nanowire 19 is possible through the change in the fluorescence intensity that corresponds to the difference between these states A and B, the rate of change in the fluorescence intensity, frequency properties of the rate of change in the fluorescence intensity, a cut-off frequency of the rate of change of the peak fluorescence intensity, the time constants at the leading and the trailing, etc.

The figure inserted in the left-hand side of FIG. 20 is obtained by integrating the fluorescence intensity under an AC electric field and approximating the trailing part of the fluorescence intensity to a primary exponential function. It is possible to evaluate the time constant of a nanowire 19 used for the experiment in an aqueous solution, using this approximation curve. The rate of change in the fluorescence intensity means a value obtained by dividing a value obtained by subtracting the minimum value from the maximum value of the fluorescence intensity by the intermediate fluorescence intensity (a value obtained by adding the maximum and minimum values together followed by dividing the added value by two), wherein noises are excluded.

In addition, it is also possible to evaluate the presence or absence of an evaluation object and the amount of the evaluation object present, by observing the change of the fluorescence intensity before and after the binding of the evaluation object, when there is an evaluation object that is specifically bound with the nanowire 19.

The figure inserted in the middle of FIG. 20 was obtained by keeping applying an AC electric field for a long duration of 10 hours. No degradation in signals are observed, and it is possible to obtain data with a good reproducibility for a long time. When one cycle of an AC electric field is regarded as one measurement, one hundred repetition of the same measurement is possible in one second when the frequency of the AC electric field is arranged to be 100 Hz for the measurement. There is no comparable devices and methods that perform evaluation in such a manner of measurement. It has been thought that degradation of fluorescence-labeled parts or nanowires will occur when nanowires using molecules or the like are employed, and the fact that such a strong bond that can endure so many times of measurement, can be obtained, is an novel finding that has not been known at all. As will be described later, experimental conditions have been found that can endure application of an AC electric field for 10 hours or longer (7,200 times of measurement or more). As a result, repeated measurement was possible. A frequency ranging from a small value of 0.2 Hz or less to about 1,000 Hz or larger is possible for the AC electric field to be used.

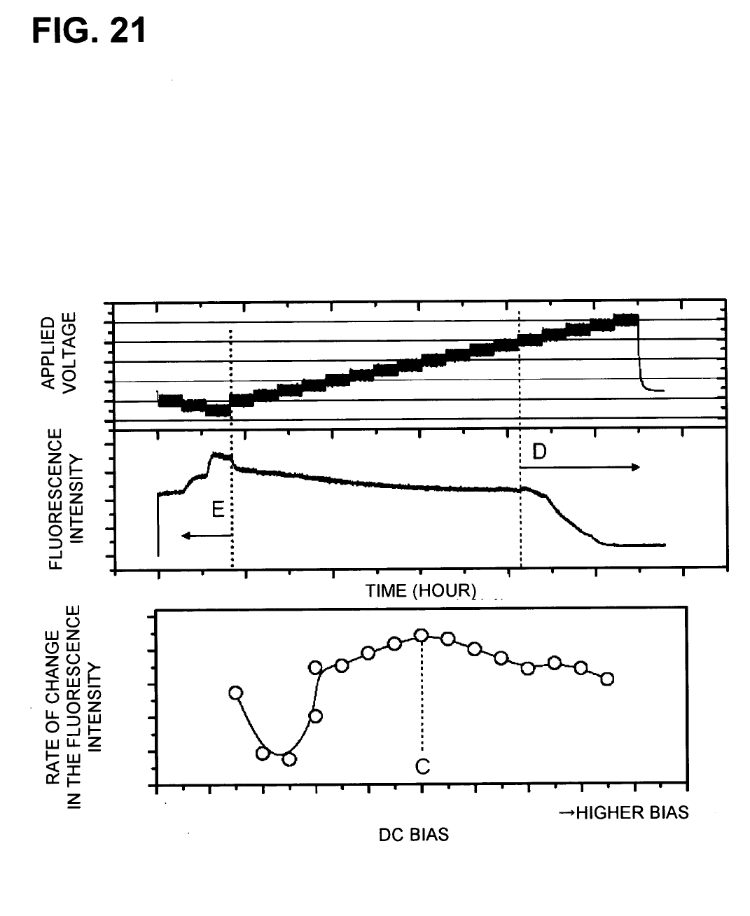

FIG. 21 shows the change in fluorescence intensity and the changing rate of the fluorescence intensity, when an AC electric field obtained by adding a weak AC bias to a stepwisely increasing DC bias is used. A sine-wave or rectangular-wave AC electric field can be applied.

From this view, it is possible to observe the difference of fluorescence intensities at respective DC biases. In particular, since the effect of the AC biasing becomes a maximum as shown in the lowest graph when the electrode is regulated to have a zero electric potential, it is possible to set a DC bias providing a maximum rate of change in the fluorescence intensity as a zero potential for the electrode. Here, it is to be noted that an electrode having a zero electric potential means a state that an electrode to which a nanowire is fixed, has an electric potential with a zero potential difference as against the counter electrode existing in a solution as a medium for an analyte. Usually, an electrode to which a nanowire is fixed, has an electric potential that is positive or negative as against the counter electrode existing in a solution as a medium for an analyte. Making the electrode have a zero electric potential means providing the electrode to which a nanowire is fixed, with a potential to compensate the potential difference. For example, if an AC electric field of ±0.2 V with 0 V at its center is applied to an electrode having an electric potential of −1 V as against the solution, the electric potential of the electrode actually shows an AC electric field between −0.8 V and −1.2 V. This means applying a negative electric field in any case. In such a case, when an offset electric potential of +1 V is applied to the electric potential of the electrode, the electric potential that makes the center of the AC electric field is regulated, resulting in a state that when an AC electric field of ±0.2 V having its center at 0 V is applied to the electrode, the electric potential of the electrode also indicates an AC electric field of ±0.2V having its center at 0V.

In the figure, the electric potential C corresponds to a zero electric potential for the electrode, and an AC bias can be applied to a nanowire at the highest efficiency, by setting the AC bias with its center at this electric potential. It was found that evaluation at a high sensitivity was thus possible.

In addition, fluorescence signals decrease rapidly and then disappear from the observation, at an electric potential higher than D in the figure. It is considered that the disappearance is caused by the degradation of the fluorescence-labeled part and the nanowire. Furthermore, at an electric potential not higher than E in the figure, a stronger fluorescence signal that is thought to be caused by the detachment of the nanowire from the carrier body is observed. This behavior is not suited for continuous evaluation with an AC bias. Therefore, it is thought important for evaluation with a high reliability with little degradation to set the electric potential in this range of D-E, that is, in a range between the lower electric potential range that appears when an electric potential to be the center of an AC electric field is changed in a stepwise manner or continuously, in which strong fluorescence signals begin to appear, and the higher electric potential range where the fluorescence signals decrease rapidly.

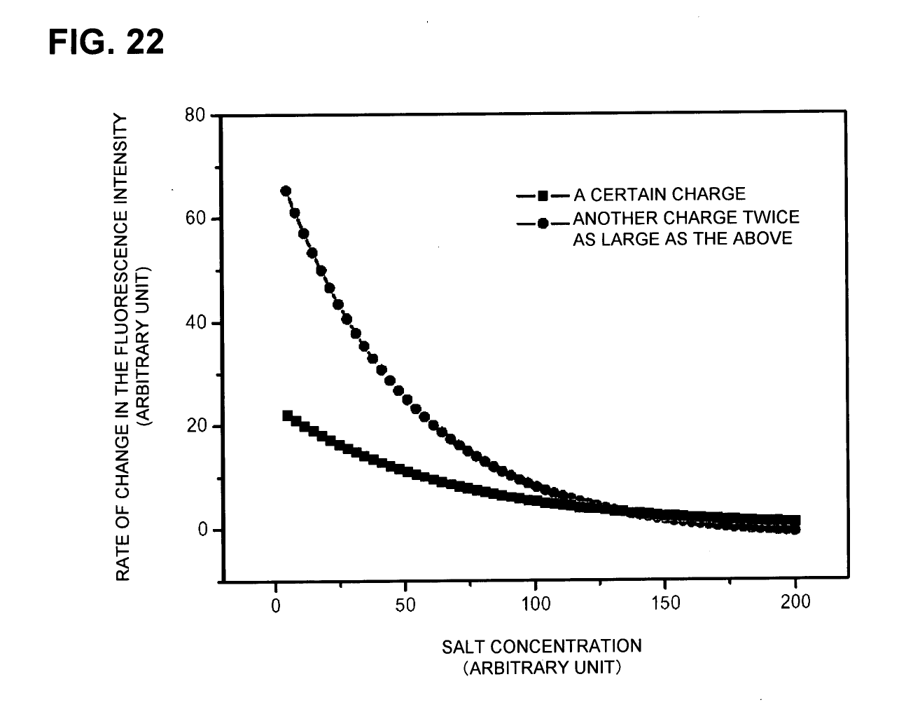

FIG. 22 shows a result of observation of the rate of change of fluorescence intensity, when the salt concentration in a medium is changed. In a range of a relatively high salt concentration, the electric charges of the charged nanowires are compensated by the salt, and therefore, are hard to be influenced by an AC electric field. In addition, two types of wires with different electric charges are used in the figure. One type of wire with twice the charge amount for the other is more strongly affected by an AC electric field, and the rate of change is larger. From the figure, it is understood that evaluation with a higher sensitivity is possible when the salt concentration is kept low, and wires that are adjusted to have a larger amount of electric charge are used. Wires with a large amount of electric charge can be realized through use of ionic polymers having a large amount of electric charge or double-stranded nucleotides, for example.

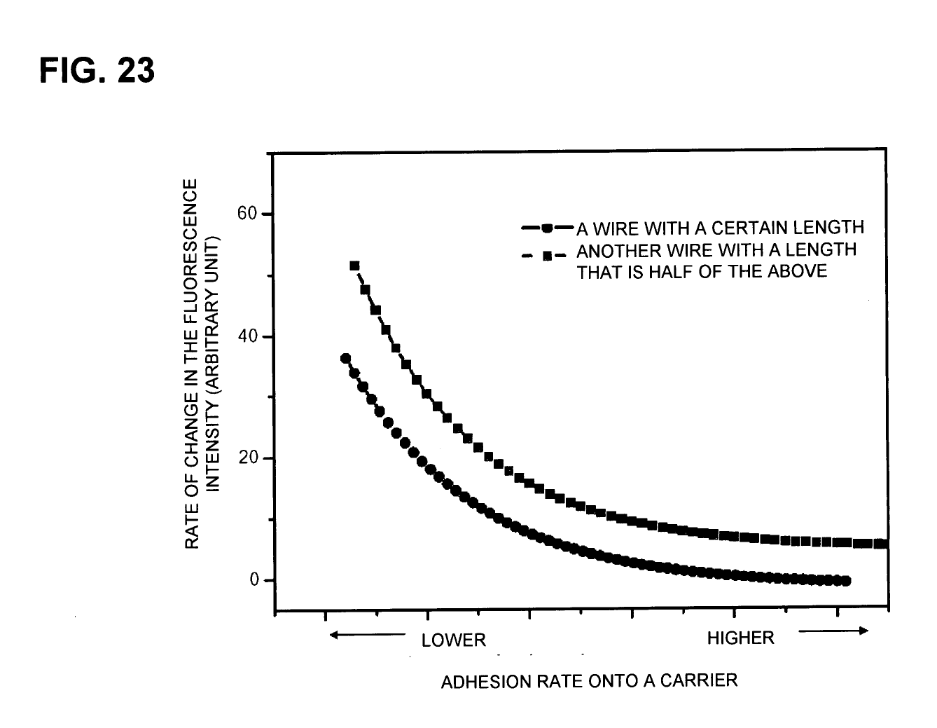

FIG. 23 shows a result of observation of the rate of change of fluorescence intensity, when the adhesion rate of charged nanowires to an electrode (in other words, adhesion rate onto a carrier body) is changed. In the range where the adhesion rate to a carrier body is high, steric hindrance is caused by the mutual interfere of the wires, and the rate of change of fluorescence intensity is not high. In the figure, two types of wires with different lengths are used. As longer wires are used, the steric hindrance occurs in a range down to a lower adhesion rate. The figure indicates that evaluation with a higher sensitivity is possible when relatively shorter wires are used at a lower adhesion rate. Here, the relatively shorter wires are those having a length of about 5 nm. It is to be noted here, that the rate of adhesion can be determined by any known methods. Representative are a method in which elements involved only in the attached molecules are measured and compared by XPS (X-ray Photoelectron Spectroscopy), a method in which molecules to be attached are labeled with a radioactive element (such as 32P), or parts of molecules to be attached are replaced with radioactive isotopes followed by measurement of the radiation for determining the adhesion rate, and a method in which the charges of nanowires are compensated with a redox marker having a positive electric charge, the electric charge necessary for the compensation is calculated through the actual measurement of the reduction current of the redox marker, and the adhesion rate of the molecules attached to the surface is calculated (see Analytical Chemistry, vol. 70, p. 4670-4677, 1998, for example).

According to the present invention, it is also possible to perform the evaluation without introducing fluorescence-labeled parts or radioactive materials into the evaluation objects. Evaluation for a tiny amount of sample is also possible. Evaluation with a high sensitivity is also possible. It is also possible to perform the evaluation, even if there are various kinds of evaluation objects in a mixed state in a sample. Furthermore, miniaturized, complex, and integrated analyte evaluating devices and excellent methods for evaluating an analyte using the devices can be realized.

The analyte evaluating device realized by the present invention can be used, as a protein detecting device to see that part of a series of protein interaction networks from an insulin acceptor to a glycogenase is decreased or increased, for example, when the hepatic cell changes the intracellular glycogen metabolism, responding to the reception state of insulin in diabetes mellitus.

Accordingly, by using such a protein detecting device, it is possible to comprehend the population of proteins, including so-called post-translational modifications such as phosphation and glycosilation.

In addition, it is possible, for example, to see that a functional degradation of a specific protein in relation with the interaction network is the cause of the defective glucose metabolism, instead of the conventional approach to see a phenomenon appearing as symptoms as a whole and correlate it with diabetes mellitus. This will make it possible to provide an appropriate diagnosis and treatment corresponding to the cause of the functional incompetence, and an appropriate verification of the result of treatment. Beside diabetes mellitus, the same procedure may be applicable to high blood pressure, hyperlipidemia, cancer (imperfect cell growth control) and other multifactorial diseases in general.

### EXAMPLES

The present invention is further explained in reference to the following examples.

### Example 1

A single-stranded oligonucleotide with a fluorescent-pigment label introduced to the 5′ terminal was synthesized and reacted with a polished Au electrode at room temperature for 24 hours so as to make the single-stranded oligonucleotide bound to the Au electrode as shown in FIG. 9. The fluorescent-pigment label may be introduced to both ends of the single-stranded oligonucleotide. It may also be introduced to a 3′ terminal of the chain. Oligonucleotide strands were kept on the Au electrode in a circular shape having a diameter of 1 mm. This fluorescent pigment was activated by light, extinguished the fluorescence when it was sufficiently near the metal surface, and emitted fluorescence when it was sufficiently distant from the metal surface.

The light irradiation device was placed at an angle of 45° from the Au electrode and set to allow incident rays to reach all over the electrode surface. In addition, the light-receiving optical fibers (a fluorescence detection device) for use had a diameter (an inner diameter of 1 mm) which was on the same level as the diameter of the electrode.

With the above-described constitution, the electrode to which oligonucleotide strands were bound was immersed into an aqueous solution, and a DC electric field with modulations such as a pulse was applied to the oligonucleotide strands by means of the two-electrode method. The fluctuation of the fluorescence intensity was measured, as the fluorescent pigments on the oligonucleotide strands were activated with a UV lamp to emit fluorescence. A solution obtained by adding 10 mM of a tris buffer solution (2-amino-2-hydroxymethylpropane-1,3-diol) and 280 mM of sodium chloride as a salt to pure water, was used. It is to be noted that the three-electrode method may be applied instead of the two-electrode method.

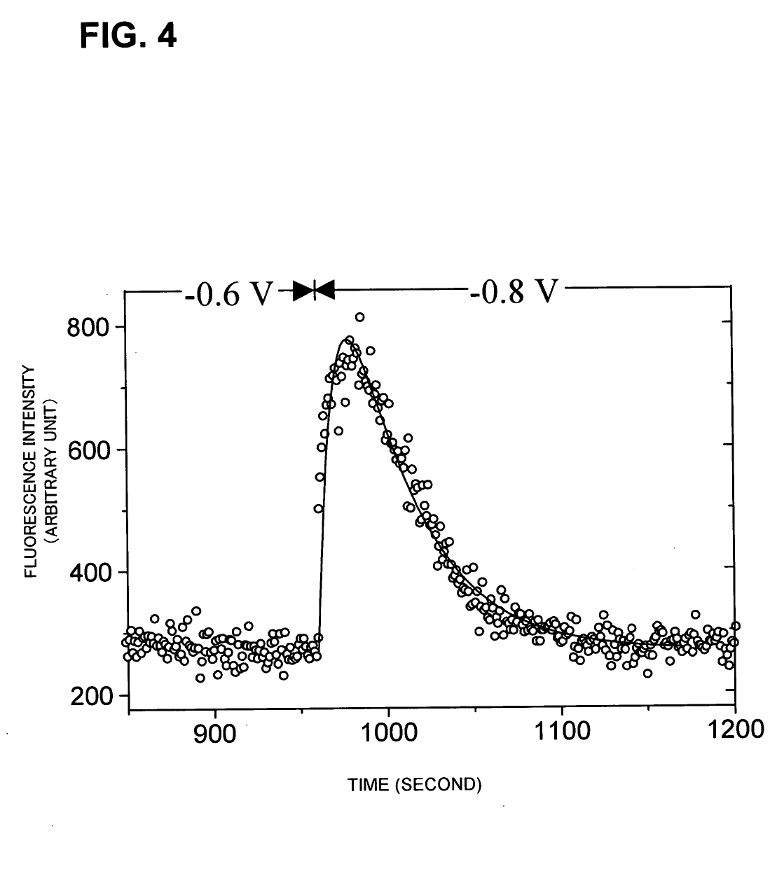

FIG. 4 shows a change of fluorescence intensity as a negative electric field of −0.8 V was applied to the electrode. It is considered that oligonucleotides that were anions were detached from the electrode by Coulomb repulsion, and accordingly, the fluorescent molecules bound to the oligonucleotides moved away from the electrode, with the result that fluorescence that had been extinguished owing to the location of the fluorescent molecules that were in the vicinity of the electrode, started to emit fluorescence.

Since the size of the electrode (the surface area of a carrier) and the fluorescence detection area that was determined by the inner diameter of the light-receiving optical fibers were on the same level, oligonucleotides that were distant from the electrode went out of the light detection area by diffusion quickly. Accordingly, decrease in fluorescence was observed. Outlined circles in FIG. 4 indicate examples of data of fluorescence intensity observed for the present exemplary embodiment. Solid lines in FIG. 4 indicate the result of calculations obtained by using the above-described equation (3). They coincide with the experimental results very well. The time constant at detachment τᵣ₁, the time constant at diffusion τ_{d1}, and the relative amount of the oligonucleotides Nc, that were first bound to the electrode were 9.4±0.7 seconds, 38.3±2.4 seconds, and 803±28 (arbitrary unit), respectively.

### Example 2

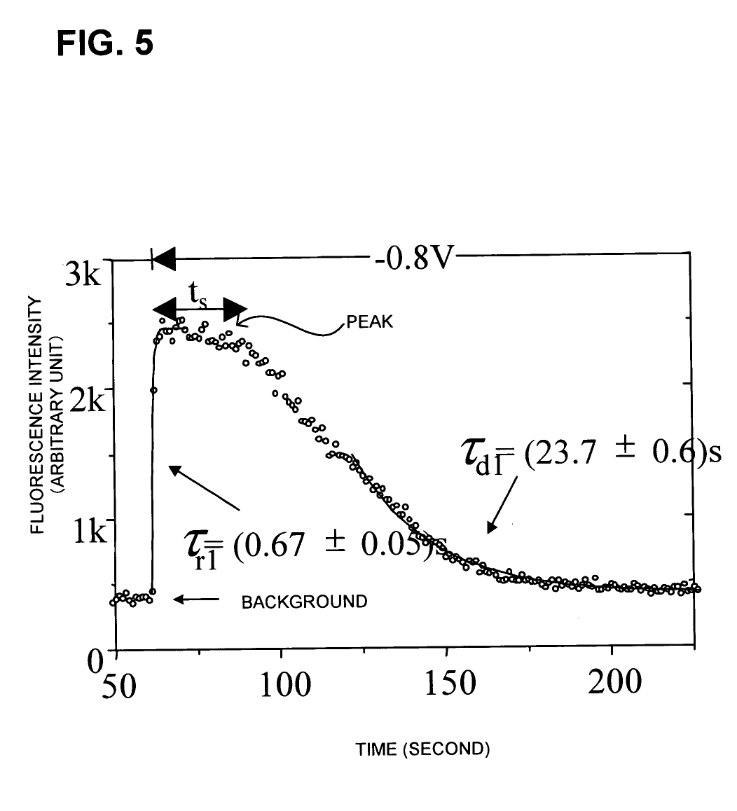

The same measurement was conducted as EXAMPLE 1 except that the diameter of the Au electrode was 2 mm, twice the size of the one in example 1, as shown in FIG. 12, that was sufficiently large as compared with the inner diameter of the light receiving optical fibers. The results are shown in FIG. 5.

In this case, the detachment of oligonucleotides occurred similarly as the electric field was applied, and the increase of fluorescence intensity was observed. After that, since the diameter of the Au electrode was sufficiently larger than the inner diameter of the optical fibers, diffusion around the light-receiving optical fibers was retarded, with the result that a time tₛ for the fluorescence to be saturated appeared. Then, as time went by, the oligonucleotides were diffused to the surroundings, and decrease of the fluorescence intensity was observed.

Since the oligonucleotides that had been once electrostatically repulsed spread out of the light detection area by diffusion after sufficient elapse of time, it was possible to observe the detachment process by electrostatic repulsion and the diffusion process to the outside, separately. The outlined circles in FIG. 5 are examples of data of fluorescence intensity observed in this example. The solid line in FIG. 5 shows the result of separate evaluations of the time constants at the detachment and at the diffusion. The experiment results and the approximation show very good agreement. The time constant at detachment τᵣ₂ and the time constant at diffusion τ_{d2} were 0.67±0.05 seconds and 23.7±0.6 seconds, respectively.

### Example 3

The measurement of the change of fluorescence intensity was conducted using the same setup as the one employed for EXAMPLE 1 except for the electric filed applied to the electrode and the salt concentrations.

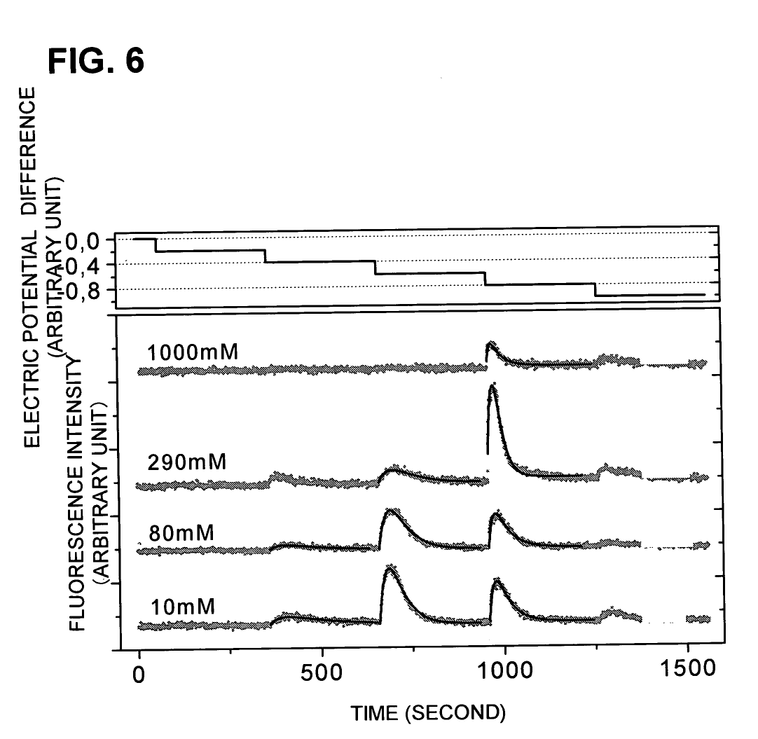

Various kinds of DC negative electric fields (−200 mV, −400 mV, −600 mV, −800 mV, and −1000 mV) were applied to the electrode, and the salt (NaCl) concentration was varied (0 mM, 70 mM, 280 mM and 990 mM), with a constant tris buffer concentration of 10 mM. The change of fluorescence intensity is shown in FIG. 6. The total salt concentrations were 10 mM, 80 mM, 290 mM, and 1,000 mM.

At 10 mM and 80 mM salt concentrations, a relatively large fluorescence intensity was observed from a relatively low electric field of −600 mV. A maximum fluorescence intensity was observed at a point of negative electric field of −800 mV at a salt concentration of 290 mM.

From this, it is understood that it is possible to observe the fluorescence from a relatively low electric field, and to perform measurement in which the effect of the electric field is suppressed to a minimum, by adjusting the salt concentration to 10 mM or 80 mM. Also, it is understood that it is possible to observe the fluorescence with a relatively high sensitivity, by setting the salt concentration at 290 mM and the electric field at −800 mV.

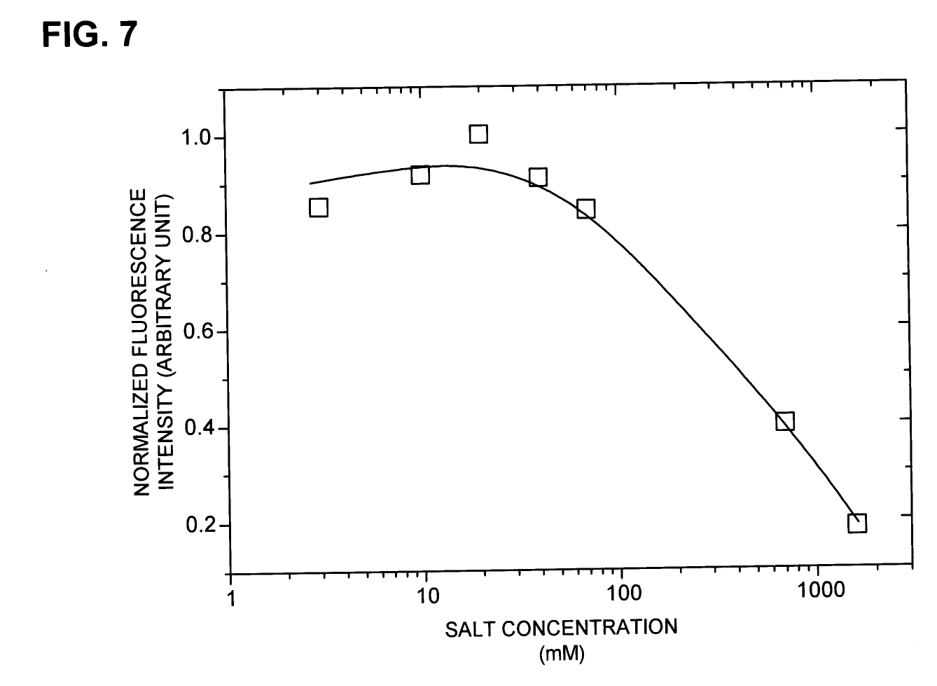

Furthermore, FIG. 7 shows the relationship between the normalized fluorescence intensities (the values obtained by subtracting the background from the peak value of fluorescence intensity in FIG. 5) and the salt concentrations wherein the same setup as the one for EXAMPLE 2 was employed, the negative electric field applied to the electrode was fixed to −800 mV, and the salt concentration was varied finely (3 mM, 10 mM, 20 mM, 40 mM, 70 mM, 700 mM, and 1, 600 mM). Only the tris buffer agent was employed for making 3 mM and 10 mM concentrations. The rest conditions were prepared by adding specific amounts of salt with a constant concentration of the tris buffer agent at 10 mM.

From FIG. 7, it is clear that the peak value of fluorescence intensity takes a large value at a specific salt concentration or less, or more specifically at 100 mM or less, and it is possible to realize the detection sensitivity of the device using an analyte at a sufficiently high level, by regulating the salt concentration in this range.

In EXAMPLES 1, 2 and 3, the change in signals by the binding of an evaluation object to an analyte was not investigated. However, since the detachment and diffusion from an electrode is greatly dependent on the electric charge and mass of a substance bound to the electrode, it is considered that when, for example, an antibody or the like (evaluation object binding part) that has a specific affinity towards a specific protein (evaluation object) is made to be bound to one end or both ends of the oligonucleotides used in the examples, and the specific protein is bound specifically to this antibody or the like, change in properties related with the detachment and/or diffusion that are dependent on the electric charge and mass of the protein will appear, with the result that the detection of the protein is made possible.

### Example 4

A single-stranded oligonucleotide with a fluorescent-pigment label introduced to the 5′ terminal and a thiol (SH) group to the 3′ terminal was synthesized and the thiol group was reacted with a polished Au electrode at room temperature for 24 hours so as to make the single-stranded oligonucleotide bound to the Au electrode as nanowires. The single-stranded oligonucleotide may be formed from one residue or more. The fluorescent-pigment label may be introduced to an intermediate part of the single strand. It may also be introduced to the 3′ terminal of the chain. When it is introduced to the 3′ terminal, the thiol group may be introduced to the 5′ terminal. The oligonucleotide strands were kept on the Au electrode in a circular shape having a diameter of 2 mm.

The fluorescent pigment had a property that it was activated by light, was made to extinguish or reduce fluorescence emission by the quenching effect when it was sufficiently near the electrode surface, and was made to increase the emission when it was sufficiently far from the electrode surface. The optical fibers for the incident light were placed at an angle of 45° from the Au electrode and set to allow the incident rays to reach all over the electrode surface. In addition, the light receiving optical fibers had a size which was on the same level as the diameter of the electrode.

The above-described setup was employed. The electrode with oligonucleotide strands fixed thereon was immersed in an aqueous solution of electrolytes. A rectangular-wave AC electric field was applied to the oligonucleotide strands by the two-electrode method. The fluorescence pigments on the oligonucleotide strands were activated with a UV lamp to emit fluorescence. The fluctuation of fluorescence intensity was measured.

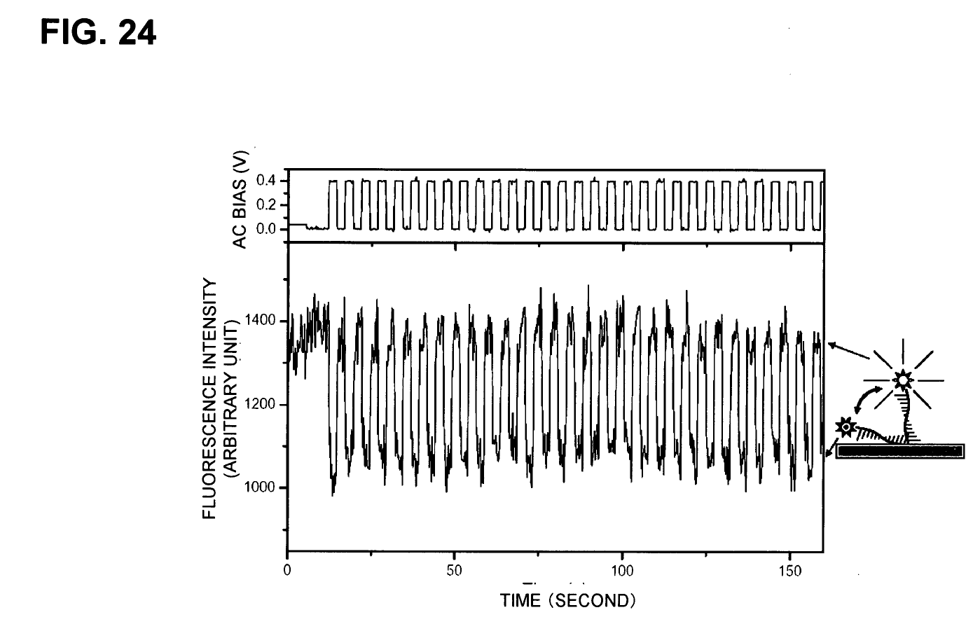

A rectangular-wave AC voltage of +200 mV with 200 V at its basis (0-400 mV) was applied to the electrode. The result of the observation of the fluorescence intensity fluctuation is shown in FIG. 24. As the right-hand side part shows, the nucleotides having negative charges are detached from the electrode by electrostatic repulsion, when the electrode potential is set relatively negative in the electric potential, and the fluorescence pigments bound to the nucleotides are detached from the electrode. As a result, the fluorescence of the fluorescence pigments that have been extinguished or reduced in intensity owing to their locations in the vicinity of the electrode, is emitted or increases in intensity. In the case of a positive potential, the fluorescence pigments are attracted by the electrode through electrostatic attractive force, and the fluorescence is made to be extinguished or reduced in intensity by the quenching effect.

The rate of change (referred to as modulation, hereinafter) of fluorescence intensity by the AC voltage is strongly dependent on the moving phenomenon of nucleotides in aqueous solutions, and the behavior of nucleotides in an aqueous solution can be evaluated, by analyzing the time constant of modulation, the width of modulation, frequency properties of the rate of change in the fluorescence intensity and a cut-off frequency of the rate of change in the fluorescence intensity.

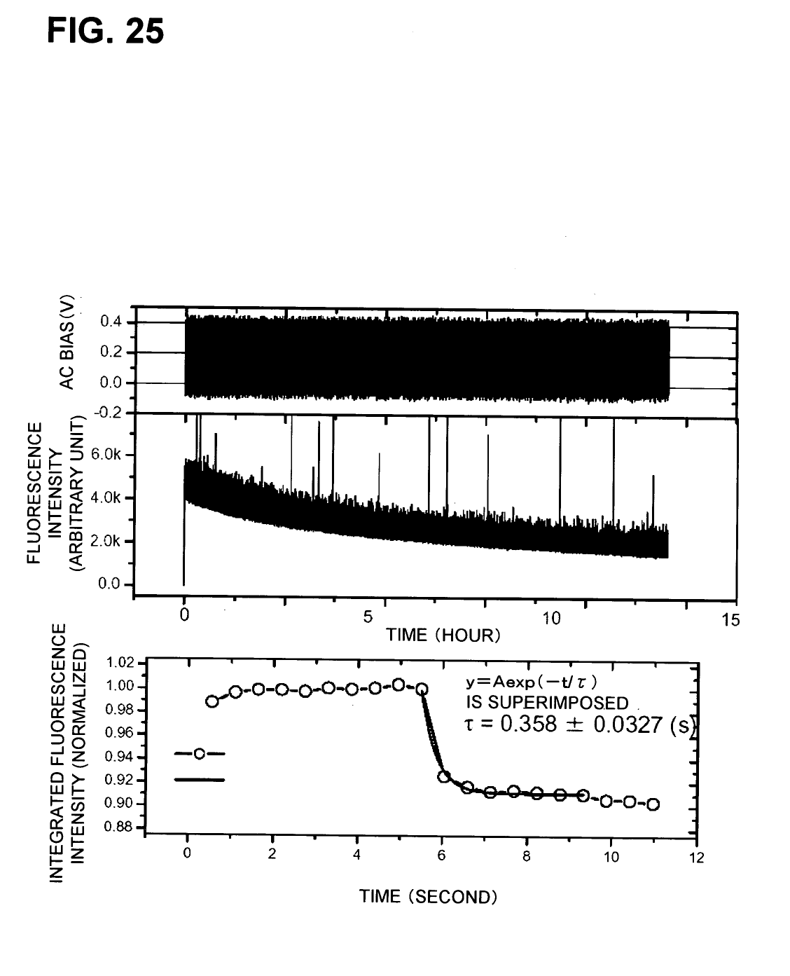

The bottom graph in FIG. 25 is an example of integrated data of modulation in which the decreasing curve of fluorescence intensity is superimposed with an exponential function. The time constant at attenuation was determined to be 0.358±0.0327 seconds. The top two graphs in FIG. 25 are results of measurement when an AC voltage was applied for 10 hours or longer. It was found that the phenomenon was observed without any degradation in the measurement for 10 hours or longer, proving that measurement with a high reliability is feasible.

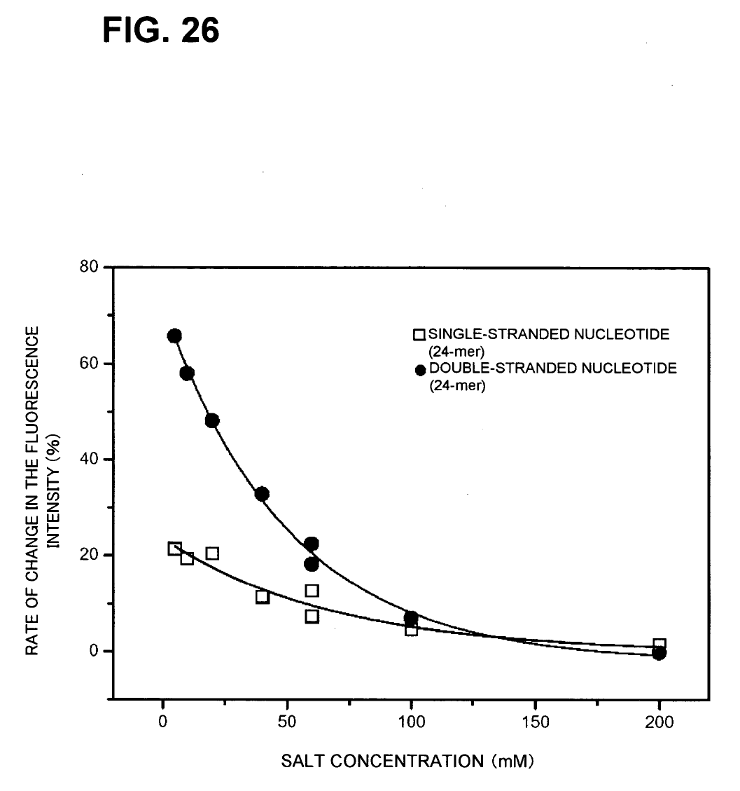

FIG. 26 shows a result of observation of the change in the modulation by changing the salt (NaCl) concentration in an aqueous solution. In a region of a relatively high salt concentration, the compensation effect of the counter ion (Na⁺ in this case) is large and is hard to be affected by an AC voltage, and accordingly the modulation is small. In the figure, the change of modulation on a double-stranded nucleotide is also shown, the double-stranded nucleotide having been formed using a complementary strand for a single-stranded nucleotide. The double-stranded nucleotide having twice as large an electric charge was influenced by an AC electric field strongly, and accordingly, indicated a large modulation. From the figure, it is understood that evaluation with a high sensitivity is possible when the salt concentration is kept low, and a nucleotide with a higher electric charge is used.

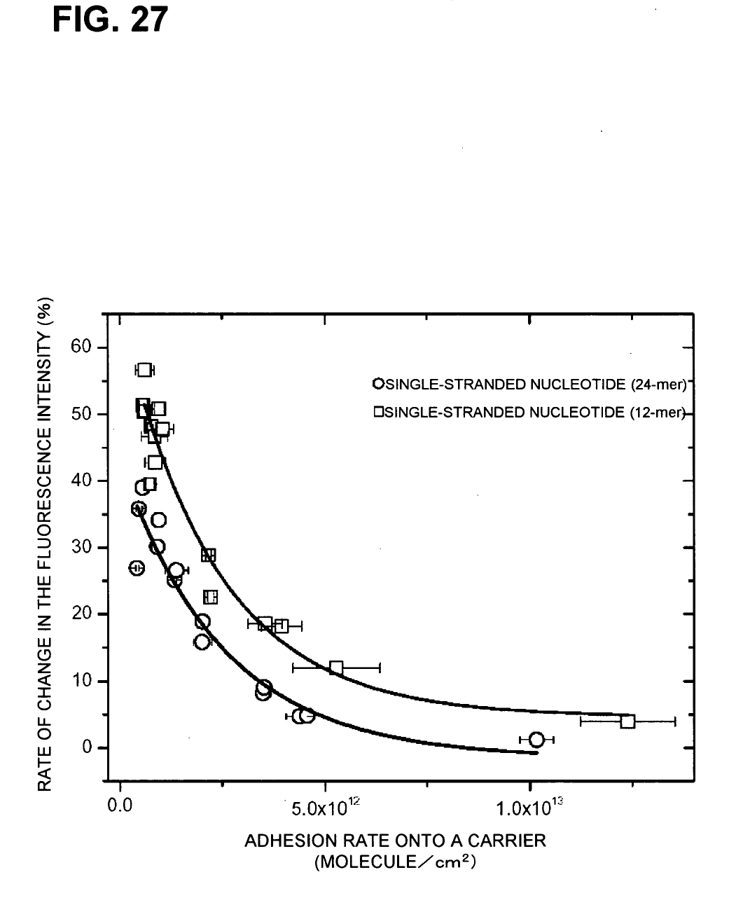

FIG. 27 shows a result of observation of the change in the modulation by changing the density of oligonucleotides on the electrode surface on which they are fixed (adhesion rate on a carrier body). In the region of a high adhesion rate on the carrier body, the modulation is small owing to the steric hindrance caused by the mutual interference between the nucleotides. Since two types of single-stranded oligonucleotides different in lengths (12-mers and 24-mers) are used as nanowires in the figure, it is understood that steric hindrance is caused at a smaller surface adhesion rate with those having the longer length. Also from the figure it is understood that evaluation with a higher sensitivity is possible when nucleotides having a relatively shorter length are used at a smaller surface adhesion rate.

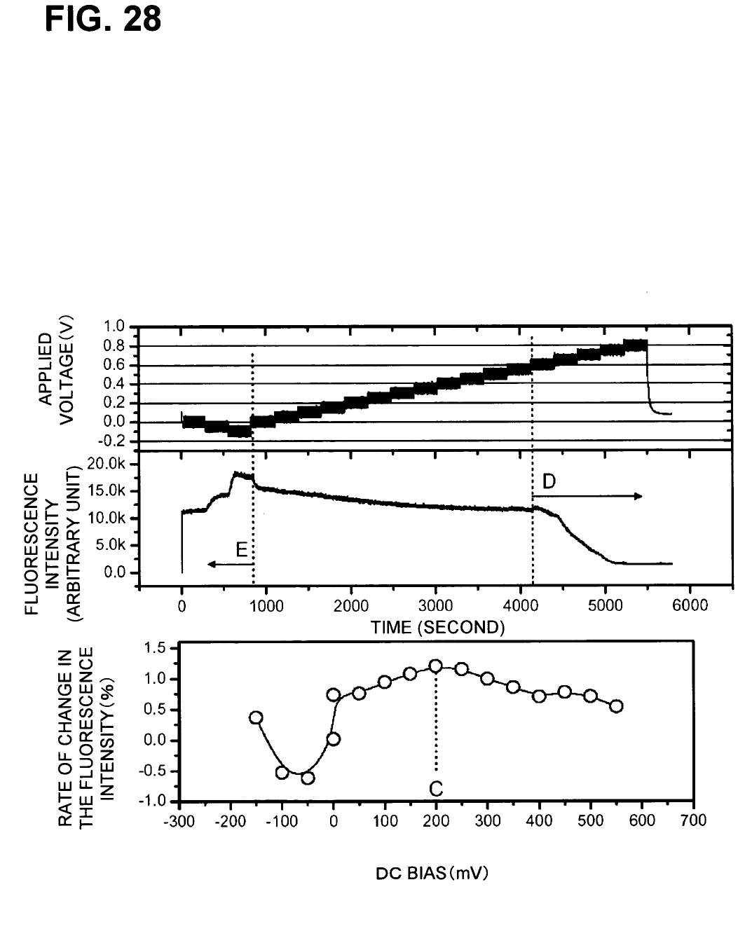

FIG. 28 shows a result of observation of the changes in the fluorescence intensity and modulation when an AC voltage obtained by adding an AC bias of ±50 mV to a stepwisely increasing DC bias was used.

From this figure, the difference in modulations can be observed at different DC biases. Particularly, since the effect of AC bias was at its maximum when an electrode had a zero electric potential, it is understood that an AC bias can be applied to the nucleotides at a maximum efficiency by setting the AC bias around this potential, with the result that evaluation with a high sensitivity is made possible.

Furthermore, in the voltage region of not less than D in this figure, the fluorescence intensity was decreased rapidly and became unobservable soon. Accordingly, it is considered that degradation of the fluorescence-labeled parts and nucleotides occur in this region. Furthermore, in the potential region of not more than E in this figure, a strong signal that was considered to be caused by detachment of nanowires from the carrier was observed. Accordingly, this region is not suited for the continuous evaluation with an AC bias. It can be considered that setting of a voltage for application to an electrode to a range of D-E is necessary for evaluation with a high reliability and without degradation.

In this example, chemical adsorption of a thiol group is applied for fixing the nucleotides onto the electrode. However, any technique is applicable as long as they can be fixed on the electrode. Utilization of the adsorption action of nucleotides themselves including physical adsorption would provide similar effects.

Furthermore, the nucleotides is used as an evaluation object in the form of charged nanowires fixed on the electrode in this example. Nanowires to which other substances with electric charges are fixed may be used as an evaluation object. The evaluation is also possible in quite the same way when substances (for example, proteins) having a property of specifically binding to nanowires, or substances (for example, proteins) having a property of specifically binding to a substance attached to nanowires (for example, antibodies), are used as evaluation objects. In such cases, continuous evaluation of the evaluation objects while applying an AC voltage as in this example may be feasible. In the case of using nanowires and substances to be attached to the nanowires in combination, evaluations of the presence or absence of the binding of evaluation objects, the amount of binding, etc. are possible by monitoring the change before and after the binding.

### Example 5

Single-stranded oligonucleotides with a fluorescent-pigment label introduced to the 51 terminal and a thiol (SH) group introduced to the 3′ terminal, and complementary-stranded oligonucleotides with biotins introduced to the 31 terminal were hybridized and the thiol group was reacted with a polished Au electrode at room temperature for one hour, to bind the resulting double-bonded oligonucleotides to the Au electrode as nanowires. One or more residues are sufficient for the single-stranded nucleotides. The fluorescent-pigment label may be introduced to an intermediate part of the single strands. It may also be introduced to the 31 terminal of the chain. When it is introduced to the 3′ terminal, the thiol group may be introduced to the 5′ terminal. In this case, the biotins on the complementary strands may be introduced to the 5′ terminal. The oligonucleotide strands were kept on the Au electrode in a circular shape having a diameter of 2 mm.

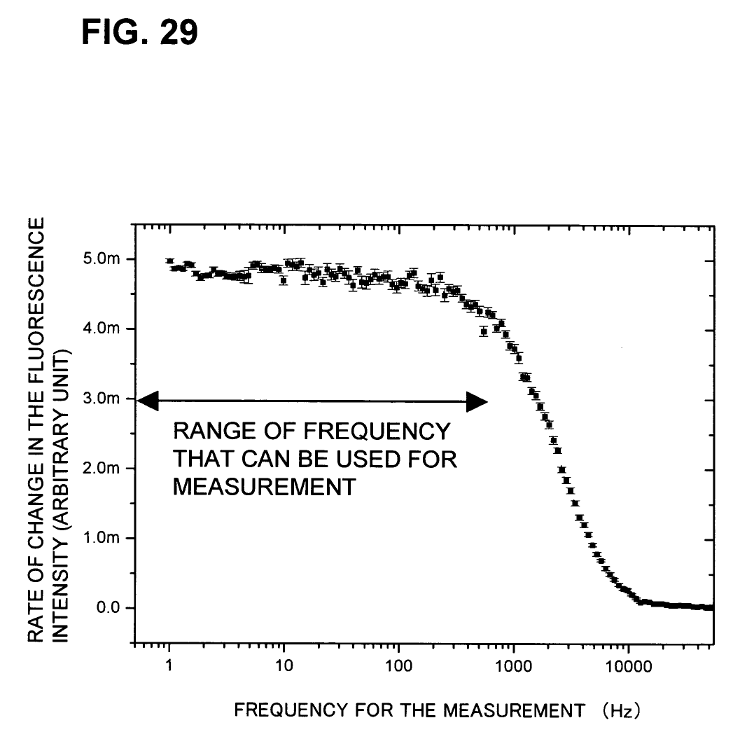

The measurement system used was quite the same as of Example 4. Instead of a rectangular-wave AC voltage, a sine-wave AC power source with an arbitrarily changeable frequency was used, and only the change in modulation was observed by a lock-in-amplifier to perform evaluation of frequencies that can be used for the measurement. The result is shown in FIG. 29. From FIG. 29, it is understood that the measurement is possible without degrading the sensitivity of the modulation up to a high frequency on the order of 1,000 Hz or more, which should contribute to a high speed measurement.

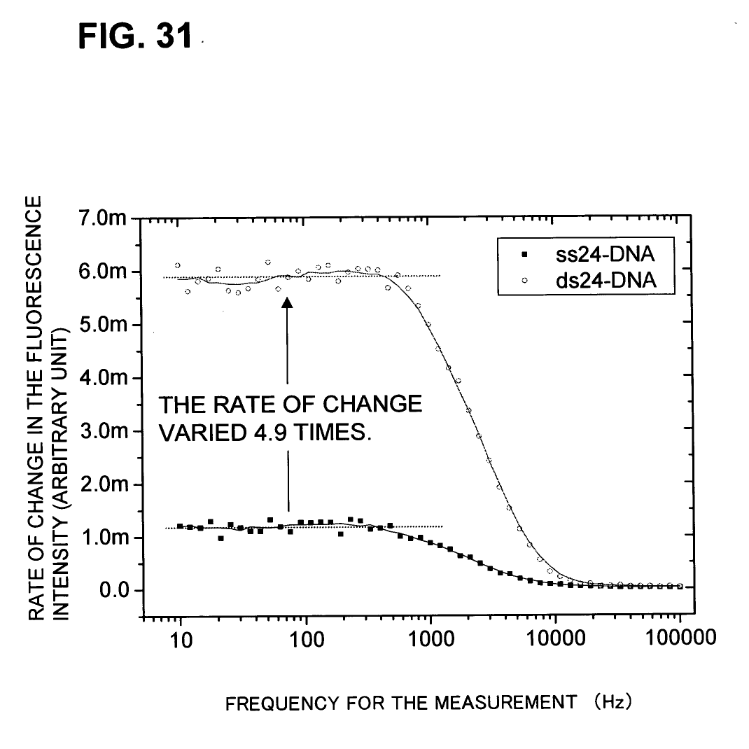

FIG. 31 indicates the frequency properties before and after the hybridization of a single-stranded oligonucleotide adhered to a Au electrode with a complementary oligonucleotide by the same adhesion conditions. The rate of change in the modulation increased 4.9 times in a wide range of frequency for the measurement of, for example, 10-1,000 Hz. Accordingly, it is understood that the hybridization was detectable in a wide range. From this, it is understood that various evaluations are possible from the frequency properties of the rate of change in the fluorescence intensity, such as the behavior of the rate of change in the fluorescence intensity when the frequency of the applied voltage is changed variously.

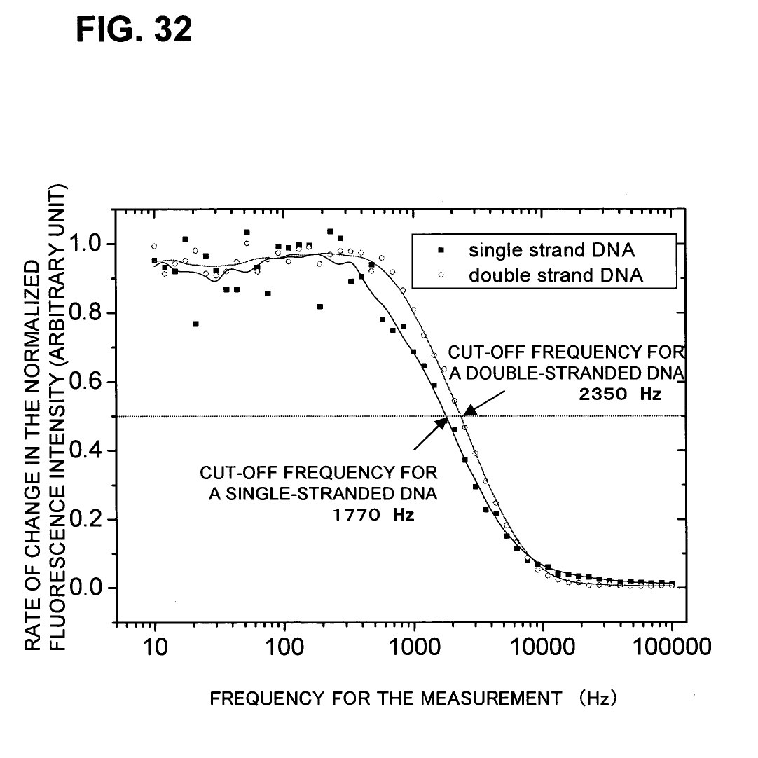

FIG. 32 indicates data from FIG. 31 that are normalized by the maximum value. It is understood that the cut-off frequency (defined as a value when the rate of change in the fluorescence intensity is half of the maximum value, here) changes from 1,770 Hz to 2,350 Hz by the hybridization. From this it is understood that measurement with a high sensitivity is made possible by paying attention to the cut-off frequency of the object for the measurement.

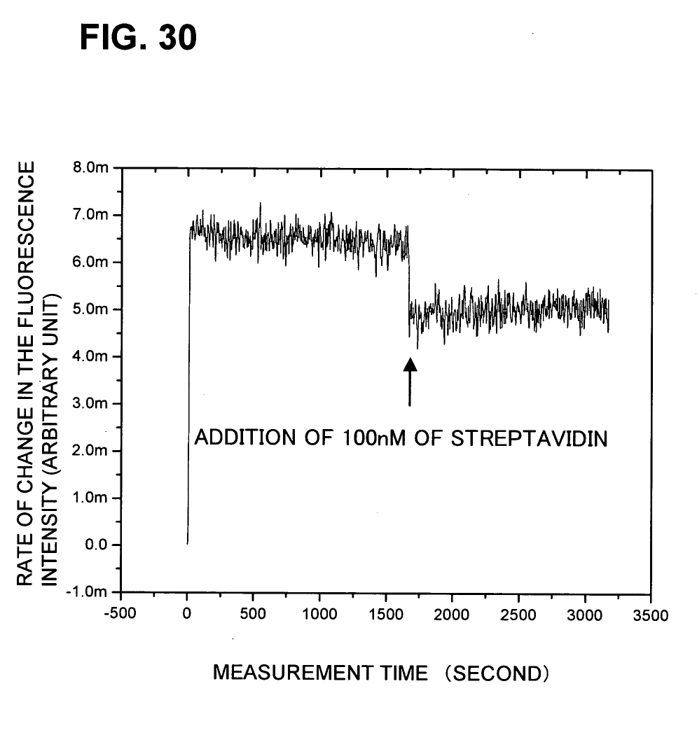

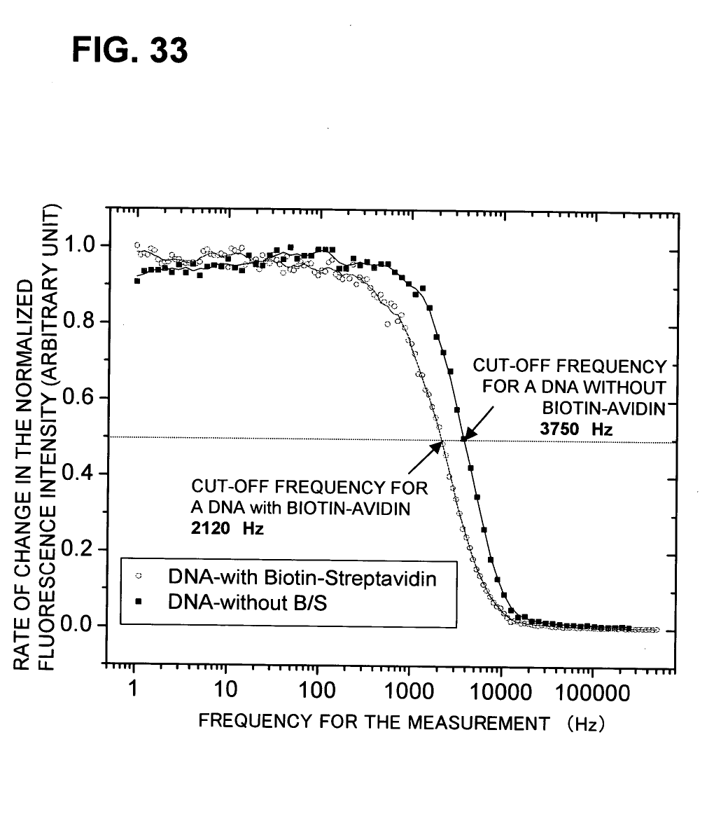

Furthermore, FIG. 30 shows a result of observation of the change in modulation, when a small amount (100 nM) of streptavidin that binds to a biotin specifically with a complementary strand introduced was added to an aqueous solution. The frequency was fixed at 37 Hz for the measurement. The signal change before and after the addition of streptavidin was measured in real time. From FIG. 30, it is understood that the specific binding of the biotin to streptavidin is clearly detected as a signal change in modulation. It is also understood that a real time detection is possible by detecting the change in the second unit. FIG. 33 indicates the change in the frequency properties in the presence and absence of a biotin-streptavidin mixture wherein the data are normalized by the maximum value. By the attachment of the biotin-streptavidin mixture, the cut-off frequency shifts from 3,750 Hz to 2.120 Hz. It is supposed that the adhesion of the mixture increased the whole molecular mass, making it difficult for the whole molecule to make a movement at a higher frequency. Accordingly, the change in the whole mass influences the cut-off frequency, and from this, it is clear that the presence of absence of an object adsorbed to a nanowire can be detected by paying attention to the cut-off frequency.

While, in this example, hybridization of oligonucleotides and the biotin-streptavidin specific binding were used for the detection, it goes without saying that other type of measurements including those for antigen-antibody reactions, specific bindings between all kinds of substances, as well as partial specific bindings wherein a SNP (single nucleotide polymorphism) type complementary DNA is hybridized, are possible.

In a narrow sense, analyte evaluation devices and methods for evaluating analytes realized by the present invention can be utilized in detection of biopolymers which now attracts much attention. Furthermore, by utilizing optimum device setups according to the present invention, not only detection of biopolymers, but also comprehension of electric properties, diffusion properties. etc. of biopolymers and artificial nano structures, is feasible, which will lead to applications in medical fields. It is expected that the applications will become wider, as the functions of biomaterials obtained through the human genome project are elucidated.

1. An analyte evaluating device comprising:
a carrier body that can be bound with an analyte having a fluorescence-labeled part that can emit fluorescence by light received when the distance between the fluorescence-labeled part and the carrier body is enlarged, the distance between the fluorescence-labeled part and the carrier body being variable by an external action;
a light irradiation device for the fluorescence-labeled part to emit light; and
a fluorescence detecting device for detecting the fluorescence emitted by the fluorescence-labeled part,
wherein at least one factor selected from the group consisting of a light irradiation angle, a light irradiation intensity, a light irradiation area, a fluorescence detection angle, a fluorescence detection area, the shape of the carrier body, the surface area of the carrier body, a salt concentration in a medium for use in the detection, and the adhesion density of analytes on the carrier body, is adjustable.

2. An analyte evaluating device according to claim 1 wherein the distance between said fluorescence-labeled part and said carrier body can be varied by a responding part equipped on at least one of the analyte and the carrier body.

3. An analyte evaluating device according to claim 1 wherein said external action is an electromagnetic or chemical action.

4. An analyte evaluating device according to claim 3 wherein said carrier body is an electrode and said electromagnetic action is realized by applying an electric potential difference between said electrode and a counter electrode.

5. An analyte evaluating device according to claim 1 wherein said carrier body can be chemically bound with the analyte.

6. An analyte evaluating device according to claim 1 wherein said carrier body has a Au layer on the surface.

7. An analyte evaluating device according to claim 6 wherein said carrier body has an analyte binding part bound with the Au layer via a thiol group.

8. An analyte evaluating device according to claim 1 wherein said analyte has an evaluation object binding part that has a property to specifically bind to at least one evaluation object selected from the group consisting of proteins, DNAs, RNAs, antibodies, natural or artificial single-stranded nucleotides, natural or artificial double-stranded nucleotides, aptamers, products obtained by limited decomposition of antibodies with a protease, organic compounds having affinity to proteins, biomacromolecules having affinity to proteins, complex materials thereof, and arbitrary combinations thereof.

9. An analyte evaluating device according to claim 8 wherein said evaluation object is a protein.

10. An analyte evaluating device according to claim 1 wherein said responding part can be charged positively or negatively.

11. An analyte evaluating device according to claim 2 wherein said responding part comprises at least one material selected from the group consisting of proteins, DNAs, RNAs, antibodies, natural or artificial single-stranded nucleotides, natural or artificial double-stranded nucleotides, aptamers, products obtained by limited decomposition of antibodies with a protease, organic compounds having affinity to proteins, biomacromolecules having affinity to proteins, complex materials thereof, positively or negatively charged ionic polymers, and arbitrary combinations thereof.

12. An analyte evaluating device according to claim 11 wherein said responding part comprises a natural or artificial single-stranded nucleotide, or a natural or artificial double-stranded nucleotide.

13. An analyte evaluating device according to claim 11 wherein said responding part comprises a Fab fragment or (Fab)₂ fragment of an antibody.

14. An analyte evaluating device according to claim 11 wherein said responding part comprises a fragment derived from an IgG antibody, or a fragment derived from a Fab fragment or (Fab)₂ fragment of an IgG antibody.

15. An analyte evaluating device according to claim 11 wherein said responding part comprises an aptamer.

16. An analyte evaluating device according to claim 1 wherein:
said light irradiation area is not less than the surface area of the carrier body; and
said fluorescence detection area can be in the range of 80 to 120% of the surface area of the carrier body.

17. An analyte evaluating device according to claim 1 wherein:
said light irradiation area is not less than the surface area of the carrier body; and
said fluorescence detection area can be not more than half of the surface area of the carrier body.

18. An analyte evaluating device according to claim 1 wherein said carrier body is bound with said analyte.

19. An analyte evaluating device according to claim 1 wherein a plurality of the same type or different types of carrier bodies are installed.

20. An analyte evaluating device according to claim 1 wherein a plurality of the same type or different types of analytes are installed.

21. An analyte evaluating device according to claim 4 wherein:
said carrier body is an electrode; and
said responding part is a wiry formation having a diameter of not more than 100 nm that can be positively or negatively charged, is expandable and shrinkable, and is fixed on the electrode.

22. An analyte evaluating device according to claim 21 wherein at least one factor selected from the group consisting of an applied direct-current voltage, an applied alternate-current voltage, a frequency of the applied voltage, an electric potential at the center of the AC voltage, the length of the wiry formation, and the rate of adhesion of wiry formations on the carrier body is adjustable.

23. An analyte evaluating device according to claim 21 wherein said wiry formation is fixed onto the electrode by physical or chemical adsorption.

24. A method for evaluating an analyte using an analyte evaluating device according to claim 1 wherein:
the analyte is made to be bound with the carrier body;
the distance between the fluorescence-labeled part and the carrier body is varied by an external action;
a light is emitted from the light irradiation device;
the fluorescence emitted by the fluorescence-labeled part is detected by the fluorescence detecting device; and
at least one factor selected from the group consisting of a light irradiation angle, a light irradiation intensity, a light irradiation area, a fluorescence detection angle, a fluorescence detection area, the shape of the carrier body, the surface area of the carrier body, a salt concentration in a medium for use in the detection, and the adhesion density of analytes on the carrier body, is adjusted.

25. A method for evaluating an analyte according to claim 24 wherein at least one of the ratio of the light irradiation area to the surface area of the carrier body, and the ratio of the fluorescence detection area to the surface area of the carrier body, is adjusted.

26. A method for evaluating an analyte according to claim 24 wherein:
said light irradiation area is made to be not less than the surface area of the carrier body, and
said fluorescence detection area is made to be in the range of 80 to 120% of the surface area of the carrier body.

27. A method for evaluating an analyte according to claim 24 wherein:
said light irradiation area is made to be not less than the surface area of the carrier body; and
said fluorescence detection area is made to be not more than half of the surface area of the carrier body.

28. A method for evaluating an analyte according to claim 24 wherein said salt concentration in the medium is adjusted to not more than 1 M.

29. A method for evaluating an analyte according to claim 24 wherein said salt concentration in the medium is adjusted to not more than 100 mM.

30. A method for evaluating an analyte according to claim 24 wherein said analyte is made to be bound with the evaluation object before the analyte is made to be bound with the carrier body.

31. A method for evaluating an analyte according to claim 24 wherein an electrode is used as the carrier body, and an electromagnetic action is realized by applying, between said electrode and a counter electrode, an electric potential difference having a value selected from the group consisting of a constant value, a pulse value, a stepwisely changing value and a periodically changing value, or a combination thereof.

32. A method for evaluating an analyte according to claim 24 wherein at least one physical property selected from the group consisting of presence or absence of fluorescence emission, the rate of increase in the fluorescence intensity, the rate of decrease in the fluorescence intensity, the peak fluorescence intensity, the rate of change of the peak fluorescence intensity, frequency properties of the rate of change in the fluorescence intensity and a cut-off frequency of the rate of change in the fluorescence intensity is measured.

33. A method for evaluating an analyte according to claim 24 wherein the evaluation is performed, using the relationship between at least one physical property selected from the group consisting of the rate of increase in the fluorescence intensity, the rate of decrease in the fluorescence intensity, the peak fluorescence intensity, the rate of change of the peak fluorescence intensity, frequency properties of the rate of change in the fluorescence intensity and a cut-off frequency of the rate of change in the fluorescence intensity, and at least one property selected from the group consisting of a light irradiation angle, a light irradiation intensity, a light irradiation area, a fluorescence detection angle, a fluorescence detection area, the shape of the carrier body, the surface area of the carrier body, a salt concentration in a medium for use in the detection, the adhesion density of analytes on the carrier body and an applied electric potential difference.

34. A method for evaluating an analyte according to claim 24 wherein:
said carrier body is an electrode; and
said responding part is a wiry formation having a diameter of not more than 100 nm that can be positively or negatively charged, is expandable and shrinkable, and is fixed on the electrode.

35. A method for evaluating an analyte according to claim 34 wherein said analyte is evaluated by making the wiry formation come near or move away from the carrier body by applying a sine-wave or rectangular wave alternate-current electric field.

36. A method for evaluating an analyte according to claim 34 wherein at least one factor selected from the group consisting of an electric charge of the wiry formation or a substance adhered to the wiry formation, an electrostatic capacity thereof, an applied direct-current voltage, an applied alternate-current voltage, a frequency of the applied voltage, an electric potential at the center of the AC voltage, the length of the wiry formation, and the rate of adhesion of wiry formations on the carrier body is adjusted.

37. A method for evaluating an analyte according to claim 36 wherein said electric potential at the center of the AC voltage is adjusted for the carrier body electrode to have a zero voltage.

38. A method for evaluating an analyte according to claim 36 wherein said electric potential at the center of the AC voltage is changed in a stepwise manner or continuously.

39. A method for evaluating an analyte according to claim 38 wherein said electric potential at the center of the AC voltage is changed in the range between a low electric potential region where a stronger fluorescence signal is obtained, and a high electric potential region where the fluorescence signal is rapidly weakened, both of which appear when the electric potential at the center of the AC voltage is changed in a stepwise manner or continuously.
